(19) United States
(12) Patent Application Publication
(10) Pub. No.: US 2023/0047828 A1
(43) Pub. Date: Feb. 16, 2023

(54) NIPPLE AREOLAR GRAFTING METHODS AND APPARATUS

(71) Applicant: Absolutions Med, Inc., Mountain View, CA (US)

(72) Inventors: Gregory LAMPS, Smyrna, GA (US); Daniel JACOBS, Mountain View, CA (US)

(73) Assignee: Absolutions Med, Inc., Mountain View, CA (US)

(21) Appl. No.: 17/819,800

(22) Filed: Aug. 15, 2022

Related U.S. Application Data
(60) Provisional application No. 63/260,312, filed on Aug. 16, 2021.

(51) Int. Cl. A61F 2/12; A61B 17/08

(52) U.S. Cl. CPC A61F 2/12; A61B 17/085; A61F 2002/526

(57) ABSTRACT

Nipple areolar grafting methods and apparatus are described herein where one variation of a tissue transfer apparatus may generally comprise a first support structure having an elliptical or circular configuration sized to encircle a region of tissue and defining a first opening through the first support structure, a first plurality of piercing features projecting from a first side of the first support structure, a second support structure having an elliptical or circular configuration sized to encircle the region of tissue and defining a second opening which is further configured to encircle the first support structure, and a second plurality of piercing features projecting from a first side of the second support structure such that the first and second piercing features are adjacent to one another.

### CROSS-REFERENCE TO RELATED APPLICATIONS

This application claims the benefit of priority to U.S. Prov. App. 63/260,312 filed Aug. 16, 2021, which is incorporated herein by reference in its entirety.

### FIELD OF THE INVENTION

The present invention relates to devices and methods to accelerate and improve the surgical procedure of nipple areolar grafting.

### BACKGROUND OF THE INVENTION

There are approximately 10,000 gender confirming procedures annually in the United States in which patients undergo breast removal surgery followed by grafting of the nipple-areolar complex onto the new chest contour. The process of nipple-areolar grafting is multi-step, time consuming, and tedious, constituting upwards of 50% of the operative time for the entire procedure. New technologies and methodologies are desired to improve, simplify, and reduce the time required for nipple-areolar grafting whether in gender confirmation surgery or other indications.

### SUMMARY OF THE INVENTION

A device may assist or perform one or more aspects of:
a. Establishing areolar harvest circumference
b. Incising the harvest circumference
c. Completing harvest of the nipple-areolar complex either as one component or two
d. Safely storing the graft until ready to be placed on the recipient site
e. Anchoring the complex as one component or two to the recipient site
f. Establishing compression of the graft to the recipient site to encourage graft “take” and healing
g. Allow for easy removal in the post-operative period.

Multiple members comprising the apparatus described herein may be described in the following:

1^{nd} member: Areolar Transfer Patch (ATP)
2^{rd} member: Ring Needle Assembly
3^{th} member: Needle Hub Assembly
4^{th} member: Recipient Site Template (RST)
5^{th} member: vertical axis adhesive
These members are considered to be exemplary and fewer members or additional members may be included between various embodiments.

One form of embodiment used for establishing the circumference of the harvest site involves an Areolar Transfer Patch (ATP), which constitutes a first member of material with appropriate size, shape, stiffness, and penetrability coated with an adhesive and attached to the donor site to establish the graft borders, facilitate graft harvesting, and result in the excised graft being held to the first member in its desired geometry. The ATP may be round (for example, typically 15 mm-30 mm in diameter, preferably 22 mm-25 mm in diameter) or oval of similar size. The surgeon may thin the graft as desired while attached to the ATP as a device-graft complex.

A round, cylindrical Areolar Cutter with a sharp edge on at least one side may have a similar perimeter as the first member such that it fits immediately around the Areola Transfer Patch. The device may be single-use such that a sharp cutting edge is maintained. For a round first member, the cylindrical device can be rotated until it cuts the skin. In one embodiment, a depth of the cut can be determined by either a fixed or adjustable shoulder, for example, typically at 2-6 mm. If the ATP is oval shaped, a small oscillating motion can facilitate cutting the perimeter to a desired depth.

Safely storing the graft until ready for placement at the recipient site requires protection from desiccation and inadvertent disposal. In one embodiment, the device-graft complex (first member—graft complex) is placed on a firm but moistened (typically with a physiologic or near physiologic crystalloid solution) foam or similar material in a well-identified platform or container such that it won't likely be confused with a disposable sponge or other component. Alternatively, the graft may be placed on silicone, rubber, or other resilient material that would allow needle penetration while supporting the graft. Such a resilient material may be paired with a moist gauze or other moisture absorbing/wicking material to keep the graft moist.

The next steps may occur in several variations. In one variation, a second member, or Ring Needle Assembly, composed of a circular or oval array of short needles is passed through the first member and graft into the foam and kept in place for safe storage until the recipient site is ready. In another variation, the first member and graft are transferred to the recipient site and then the second member is passed through the graft into the recipient dermis. The needles prevent shear movement of the graft but do not prevent separation of the graft from the dermis, thus making future clinic removal painless and straightforward. The needles may extend into the skin, for example, 2-8 mm.

In all cases, but particularly if the nipple is grafted separately from areolar skin, a third member (Hub Needle Assembly), which constitutes one or more needles coming from a platform fitting within the inner diameter of the array of needles of the second member, can be positioned to hold the nipple tissue in position over the recipient dermal site. Said needle(s) may variably or invariably be positioned to maintain a specific depth within the recipient dermal tissue. In another variation, the needle may have a variable or fixed “stop” platform that can be positioned on the shaft of the needle(s) to limit nipple tissue migration away from the recipient dermis. A moldable material such as cotton, fabric, sponge, or foam can be placed between the platform of the third member to the nipple tissue or the “stop” proximal to the nipple tissue in order to apply gentle compression of the nipple tissue and central areolar skin graft to the recipient dermis.

The needles emanating from both the second and third members prevent horizontal or shear motion of the graft relative to the underlying recipient dermis. They do not prevent the needles from coming out in a vertical axis (away from the chest skin). For this purpose, a fourth member, consisting of a material with adhesive, covers the combined first, second, and third (if used) members to create vertical stability and graft compression (ideally near but below capillary pressure such as 10 mm Hg). In one embodiment, the fourth member is a foam, hydrogel, film or similar material with a cutout or insert site for the combined first, second, and third (if used) members to add greater stability and help limit over compression. In another embodiment, the fourth member is a simple adhesive plastic tape similar to commercial materials such as Ioban™ (3M, St. Paul, Minn.) or Tegaderm (3M, St. Paul, Minn.).

The first member may consist of any material that is biocompatible and allows penetration of the pins from the second member. Ideally, the material is stiff enough to assist with harvesting after adhered to the donor site, soft enough to allow conforming and avoid excessive pressure points on the recipient site, and pre-sized and shaped to serve as a template. It may be a hydrogel, foam, silicone sheet, collagen, fabric, other such materials, or a multi-layer laminate or assembly, etc.

The second member may consist of a number of pins embedded in or attached to a ring made of a material such as metal, plastic, glass, or composite. The pins are ideally metallic but could be ceramic, glass, plastic, or any other material that can be utilized to pierce the skin and tissues of the body. In some cases, it may be desirable to utilize a bioabsorbable material. The ring may pair with a first member to sit at or near its circumference whether as round, oval, or other geometry. In another embodiment, the second member may consist of a thin sheet of metal in which the pins are formed by cutting or otherwise removing material from the periphery leaving behind sharp phalanges. The phalanges are then bent, e.g., 90 degrees, from the plane of the metal. In another embodiment, a sheet of metal with pins or sharp phalanges can be bent around onto itself to form a, e.g., 360-degree cylindrical or oval shape, with the pins projecting parallel to the axis of the cylinder.

In another embodiment, while less stable than previous embodiments, no needles are utilized, but a fourth member is placed over the first member and graft, serving as the only fixation. A soft, compression material may be placed between the first and fourth member.

An alternate embodiment may eliminate the Areola Transfer Patch. An Areola Cutter or scalpel is utilized to harvest a graft of the desired size and shape. The graft is placed on a ring with a plurality of needles which is designed to maintain the size and shape of the graft. Alternatively, the graft may be placed on a surface and then the ring with needles place on the graft with the needles extending through the graft. In some cases, an intermediate layer (between the graft and the ring) such as an adhesive may be placed on the graft prior to harvesting or before penetrating the graft with the needles. A recipient site is prepared by de-epithelizing a region of tissue of a similar size and shape as the graft. In some cases, it may be desirable to have the recipient site be a smaller size than the graft. Prior to de-epithelizing the recipient site, it is desirable to stabilize the recipient size by placing a ring with multiple needles around the recipient site and/or adhering an adhesive tape to the site. By stabilizing the skin around the recipient site, the tendency of the epidermis to retract away from the margin of the de-epithelized region is minimized. Both the ring with needles which penetrates the graft as well as the ring with needles that penetrates the recipient site may be secured to the underlying tissue with suture, surgical staples, adhesives, bandages, or similar constructs so that they do not fall off the tissue until the desired healing has occurred.

The invention may be appropriate for procedures other than female to male gender affirming surgery such as mastectomy reconstruction, breast reduction, or other skin graft procedures. Multiple sizes and shapes can facility other grafting indication.

One variation of a tissue transfer apparatus may generally comprise a first support structure having an elliptical or circular configuration sized to encircle a region of tissue and which may define a first opening through the first support structure, a first plurality of piercing features projecting from a first side of the first support structure, a second support structure having an elliptical or circular configuration sized to encircle the region of tissue and defining a second opening which is further configured to encircle the first support structure, and a second plurality of piercing features projecting from a first side of the second support structure such that the first and second piercing features are adjacent to one another.

One variation of a method of transferring a nipple-areola complex may generally comprise securing a substrate upon a nipple-areola complex to be removed from a first tissue site, removing the nipple-areola complex with the substrate attached from the first tissue site, piercing a first support structure through a periphery of the nipple-areola complex and substrate, positioning a second support structure upon a second tissue site, and positioning the first support structure upon the second tissue site such that the first support structure is positioned within the second support structure and the nipple-areola complex is located upon the second tissue site via the first support structure.

Another variation of a tissue transfer apparatus may generally comprise a support structure having an elliptical or circular configuration sized to encircle a nipple-areolar complex, a plurality of piercing features projecting from a first side of the support structure, and a transfer substrate configured to approximate a size of the nipple-areolar complex when positioned upon the nipple-areolar complex.

Another method for transferring a nipple-areola complex may generally comprise securing a substrate upon a nipple-areola complex to be removed from a first tissue site, removing the nipple-areola complex with the substrate attached from the first tissue site, piercing a support structure through a periphery of the nipple-areola complex and substrate, positioning a recipient site template upon a second tissue site, and securing the nipple-areola complex to the second tissue site via the support structure.

Another method for transferring a nipple-areola complex may generally comprise removing a nipple from a first tissue site, securing the nipple to a central member via one or more central piercing features projecting from the central member, removing at least a portion of areola from the first tissue site, positioning the nipple within the portion of areola via the central member, piercing a support structure through a periphery of the areola such that the support structure and central member are aligned with one another, positioning a recipient site template upon a second tissue site, and securing the support structure and central member to the second tissue site.

### BRIEF DESCRIPTION OF THE DRAWINGS

FIG. 1 shows a series of needles in a ring.

FIG. 2 shows a series of needles in a semi-transparent ring.

FIG. 3 shows a series of flat needles bent orthogonally from the inner diameter of a sheet metal ring.

FIG. 4 shows a series of flat needles bent orthogonally from the outer diameter of a sheet metal ring.

FIG. 5 shows multiple needles in a central hub designed to interface with a ring of needles.

FIG. 6 shows a ring of needles sliding down around the hub of FIG. 5.

FIG. 7 shows a cross-sectional view of the hub and ring fully assembled.

FIG. 8 shows a single needle in a central hub designed to interface with a ring of needles.

FIG. 9 shows the hub of FIG. 8 fully seated against the upper surface of a ring of needles.

FIG. 10 shows a cross-sectional view of the assembly of FIG. 9.

FIG. 11 shows a series of ‘U’-shaped needles overmolded with plastic which is then overmolded again to form a ring.

FIG. 12 shows a series of ‘U’-shaped needles overmolded with plastic which is then overmolded again to form a ring with the needles forming two concentric circles.

FIG. 13 shows a breast with an Areola Transfer Patch adhered to the areola around the nipple.

FIG. 14 shows an Areola Transfer Patch attached to the areola and nipple which have been cut away from the other tissues of the breast.

FIG. 15 shows a Ring Needle Assembly with the needles being driven through the Areola Transfer Patch and the areola/nipple graft.

FIG. 16 shows a chest site with a Recipient Site Template adhered to the skin.

FIG. 17 shows the recipient site epidermis after removal from recipient site dermis.

FIG. 18 shows a Ring Needle Assembly with the nipple/areola graft being placed onto the recipient site.

FIG. 19 shows a Ring Needle Assembly with the nipple/areola graft placed onto the recipient site.

FIG. 20 shows a Ring Needle Assembly being pressed to full contact with the tissue.

FIG. 21 shows a Nipple Cutter approaching the nipple for harvest.

FIG. 22 shows a Nipple Cutter cutting the circumference of the desired portion of the nipple for harvest.

FIG. 23 shows a cross-sectional view of a Nipple Cutter cutting the circumference of the desired portion of the nipple.

FIG. 24 shows the nipple graft being harvested.

FIG. 25 shows Needle Hub needles being driven into nipple graft.

FIG. 26 shows an Areola Transfer Patch applied to areolar tissue for situations where areolar skin is harvested separately from the nipple projection.

FIG. 27 shows an Areola Cutter approaching the areolar harvest site and Areola Transfer Patch.

FIG. 28 shows an Areola Cutter cutting the outside circumference of the areola graft.

FIG. 29 shows a Nipple Cutter cutting the insider circumference of the areola graft.

FIG. 30 shows an areola graft being removed from the breast after dissecting it from underlying tissue.

FIG. 31 shows a nipple graft with Needle Hub being positioned over an areolar graft.

FIG. 32 shows a nipple graft with Needle Hub centered in an areolar graft.

FIG. 33 shows a Ring Needle Assembly sliding over the Needle Hub and into the areolar graft.

FIG. 34 shows a cross-sectional view of a Ring Needle Assembly and Needle Hub with needles penetrating the nipple and areolar graft.

FIG. 35 shows an Areola Cutter positioned over recipient site with Recipient Site Template applied.

FIG. 36 shows an Areola Cutter cutting partial thickness skin tissue at the recipient site.

FIG. 37 shows Ring Needle Assembly, Needle Hub, and graft positioned at recipient site after completion of de-epitheliaziation.

FIG. 38 shows Ring Needle Assembly, Needle Hub, and graft with needles fully inserted into recipient site.

FIG. 39 shows a cross-sectional view of Ring Needle Assembly, Needle Hub, and graft with needles fully inserted into recipient site and compressible material packed around and over the device.

FIG. 40 shows a Nipple Transfer Patch applied to a nipple.

FIG. 41 shows the nipple graft harvested from the donor nipple with a Nipple Transfer Patch.

FIG. 42 shows the nipple graft and Nipple Transfer Patch with needle from a Needle Hub penetrating through the graft.

FIG. 43 shows a Ring Needle Assembly positioned over an areolar graft.

FIG. 44 shows a Ring Needle Assembly with needles penetrating an areolar graft.

FIG. 45 shows a Needle Hub with nipple graft being lowered into a Ring Needle Assembly with areolar graft.

FIG. 46 shows nipple and areolar grafts with needles of a Ring Needle Assembly and Needle hub penetrating the grafts.

FIG. 47 shows a Ring Needle Assembly, Needle Hub, and nipple and areolar grafts being lowered onto recipient site.

FIG. 48 shows a Ring Needle Assembly, Needle Hub, and nipple and areolar grafts in position on recipient site.

FIG. 49 shows a Ring Needle Assembly, Needle Hub, and nipple and areolar grafts in position on recipient site.

FIG. 50 shows a Ring Needle Assembly, Needle Hub, and nipple and areolar grafts in position on recipient site with needles fully penetrating the tissues.

FIG. 51 shows compressible foam being fit into and over Ring Needle Assembly on recipient site.

FIG. 52 shows compressible foam in position inside and over Ring Needle Assembly on recipient site.

FIG. 53 shows an example of a nipple upon which a procedure has been performed.

FIG. 54 shows an alternate Areola Transfer Patch (ATP) with an additional layer of more rigid plastic over a soft and flexible skin-adjacent layer, the stiffer plastic layer having a gap for the needles from the Ring to pass through without having to penetrate the rigid plastic. The ATP is about to be applied to the areola.

FIG. 55 shows a cross-sectional view of the ATP from FIG. 54.

FIG. 56 shows a cross-sectional view of an Areola Cutter having two concentric rings to cut both the internal circle (or other shape) where the nipple will be placed, and the external margin, marking the ring of recipient dermis for the areolar graft simultaneously.

FIG. 57 shows a Ring Needle Assembly intended for holding an areola graft.

FIG. 58 shows a Ring Needle Assembly intended to stabilize the tissue around the areola recipient site.

FIG. 59 shows the Ring Needle Assemblies of FIGS. 57 and 58 together.

FIG. 60 shows the Ring Needle Assemblies of FIGS. 57 and 58 from the opposite angle.

FIG. 61 is a block diagram flowchart of a method for harvesting a nipple and areola from one location and grafting it into a new location in one piece.

FIG. 62 is a block diagram flowchart of a method for harvesting a nipple and areola in two pieces and grafting the nipple and areola together onto a new location.

FIG. 63 is a block diagram flowchart of another method for harvesting a nipple and areola in two pieces and grafting the nipple and areola together onto a new location.

FIG. 64 is a block diagram flowchart of a method for harvesting a tissue such as a nipple and areola from one location and grafting it in a new location utilizing adhesive patches.

FIG. 65 is a block diagram flowchart of a method for harvesting a tissue such as a nipple and areola from one location and grafting it in a new location utilizing inner and outer rings with multiple penetrating elements.

### DETAILED DESCRIPTION OF THE INVENTION

The present invention relates to methods and apparatus for facilitating the removal of the nipple-areolar complex from a breast and the grafting of the removed nipple-areolar complex onto a new chest contour.

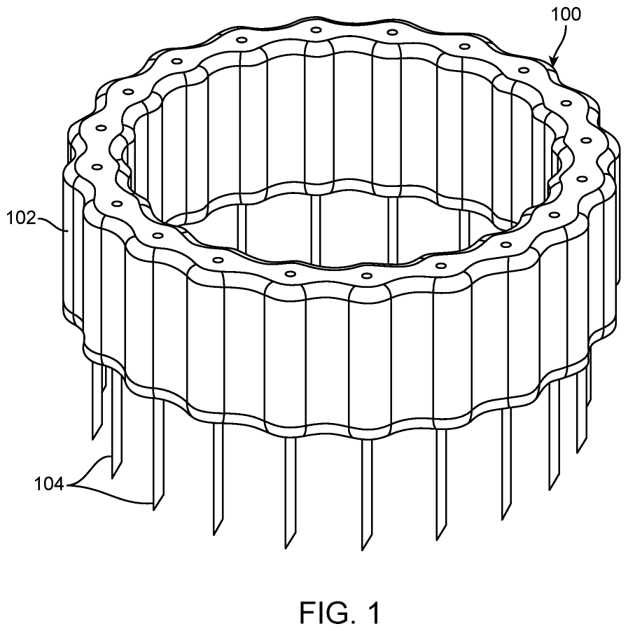

Generally, the system may include a support structure which is sized to have a circular or elliptical shape which is sufficiently large to encircle or approximate the nipple-areolar complex to be grafted. The support structure may include a circularly or elliptically shaped scaffold or support having a plurality of piercing features such as needles or staples projecting from a first side of the scaffold or support. FIGS. 1 through 12 illustrate various embodiments of the scaffold or support which is also called a Ring Needle Assembly herein.

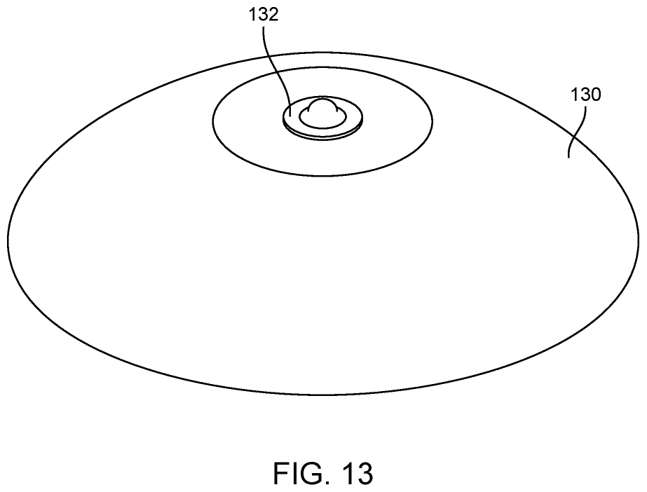

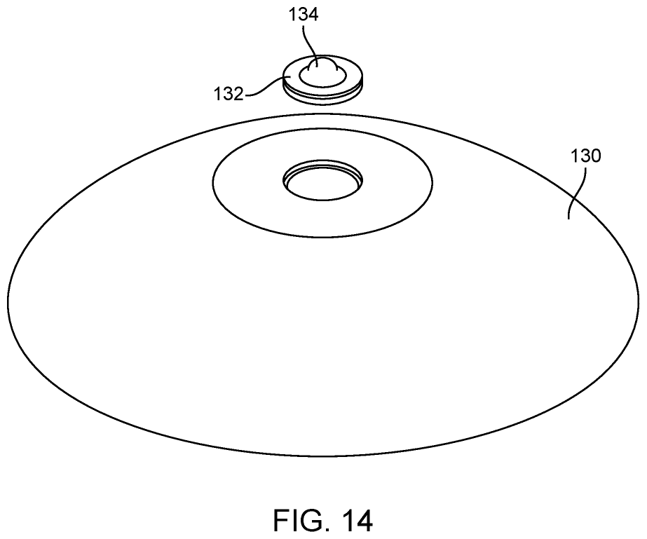

A biocompatible substrate having an adhesive surface may be sized to approximate the shape of the nipple-areolar complex and may be used with the scaffold or support. A nipple-areolar complex (134) (as shown in FIG. 14) to be removed from a breast may be first covered with this adhesive substrate called an Areola Transfer Patch (ATP) (132) to protect and maintain the nipple-areolar complex, as shown above in FIG. 13 where the adhesive substrate may be secured upon the nipple-areolar complex. The biocompatible transfer substrate may include an adhesive substrate but in other variations, the substrate may omit the adhesive such that the transfer substrate may remain adhesive-free.

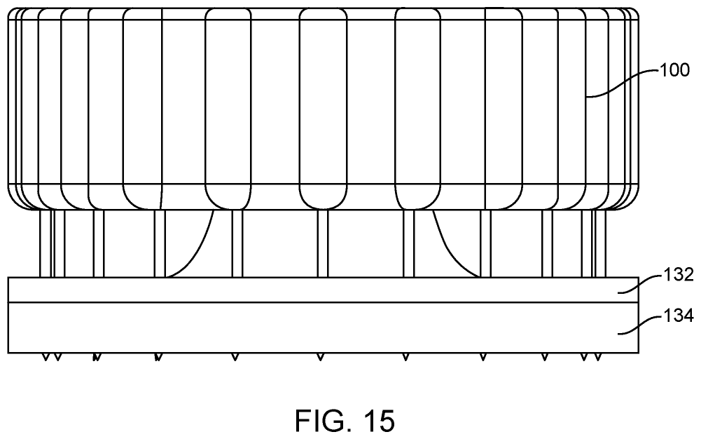

The nipple-areolar complex may be removed from the breast while remaining attached to the adhesive substrate (as shown in FIG. 14). The scaffold or structure may be then secured around a periphery of the nipple-areolar complex by piercing through a periphery of the adhesive substrate and into the tissue of the periphery of the nipple-areolar complex, as shown below in FIG. 15.

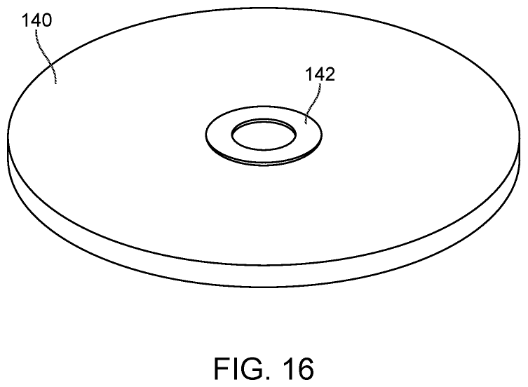

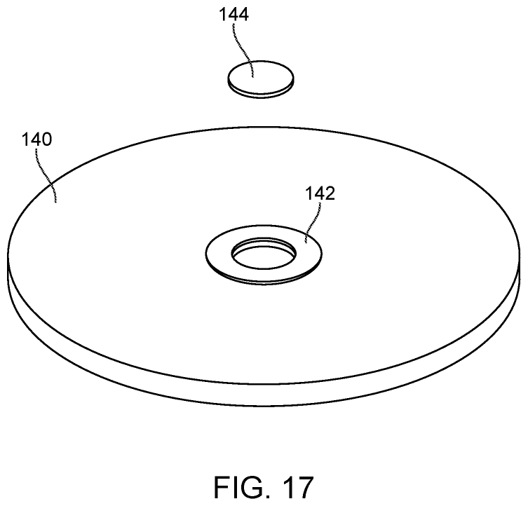

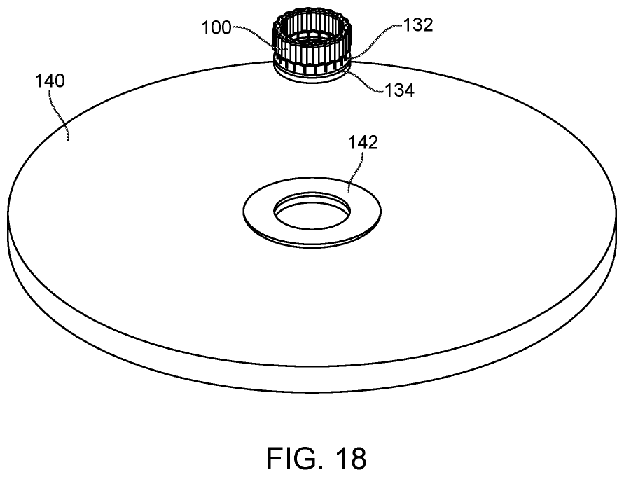

When ready for nipple transplantation, a region of the breast or chest skin (140) may be identified for the grafting of the nipple-areolar complex. Similar to the ATP, an adhesive substrate called a Recipient Site Template (RST) (142) may be used to protect and maintain the edges of the tissue around the recipient site as shown in FIG. 16. This region of the breast may have its tissue de-epithelialized (as shown in FIG. 17) and the scaffold or support having the removed nipple-areolar complex may be secured upon this de-epithelialized region by piercing the assembly via its needles or staples into the region of tissue, as shown in FIGS. 18 through 20.

While the piercing elements (104,120) such as needles or staples may prevent the nipple-areolar complex from rotating or traversing over the tissue, an additional adhesive substrate may be placed over the entire assembly to inhibit the nipple-areolar complex from being pulled away from the tissue surface.

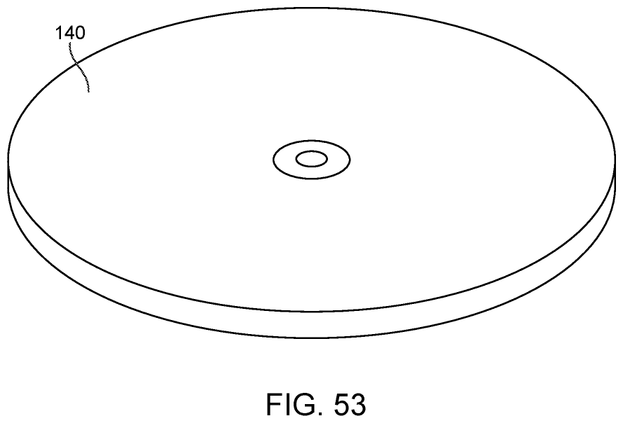

Once the nipple-areolar complex has grafted completely upon the breast tissue, the additional adhesive substrate, scaffold or support, and adhesive substrate may be removed, leaving the grafted nipple-areolar complex, as shown in FIG. 53.

The assembly and method described may be used in various applications such as subcutaneous mastectomy in TOP surgery, oncologic mastectomy reconstruction, breast reduction, or other skin graft procedures and is not limited to the procedures performed upon the breast.

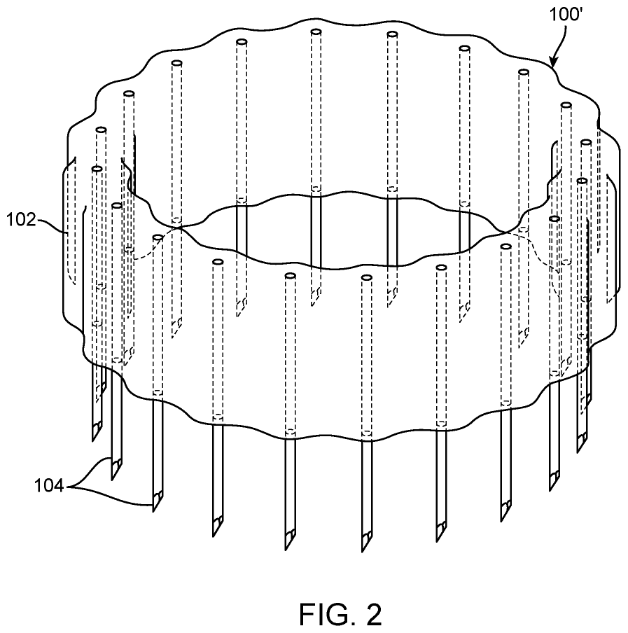

The device may include a scaffolding or support of needles arranged in a circular pattern (Ring Needle Assembly) (100) such that the needles extend or project from a first side of the scaffolding or support in parallel with sharp tips arranged in a circular, oval, or similar shape, as shown in the perspective view of FIG. 1. The needles (104) extend a distance beyond the ring (102) such that they can penetrate through the Areolar Transfer Patch (132) on the graft (134), the full thickness of the graft (e.g. 1-4 mm), and into the recipient site dermis for a short length (e.g. 2-6 mm), thus designed to prevent grafted tissue from shifting laterally or rotating on the recipient site. Such a distance is also appropriate in the absence of an Areolar Transfer Patch. It is desirable that the needles are a small distance (0.5-1.5 mm) inset from the margin of the graft. The needles may be formed from round wire, wire of a different profile, formed from sheet metal, ceramic, plastic, or any other material capable of penetrating the skin without excessive bending or breaking. Dual rows of needles may be arranged to control inside margin and outside margin of tissues or alternatively to control a wider band of tissue around the edge of the graft. While the ring (102) may be fabricated from a variety of materials, such as plastic, the ring is (102) desirably structurally robust enough to prevent the needles from flexing or moving out of alignment relative to one another. Furthermore, the ring (102) may be fabricated as an opaque, semi-transparent, or fully transparent structure (100′), as shown in FIG. 2.

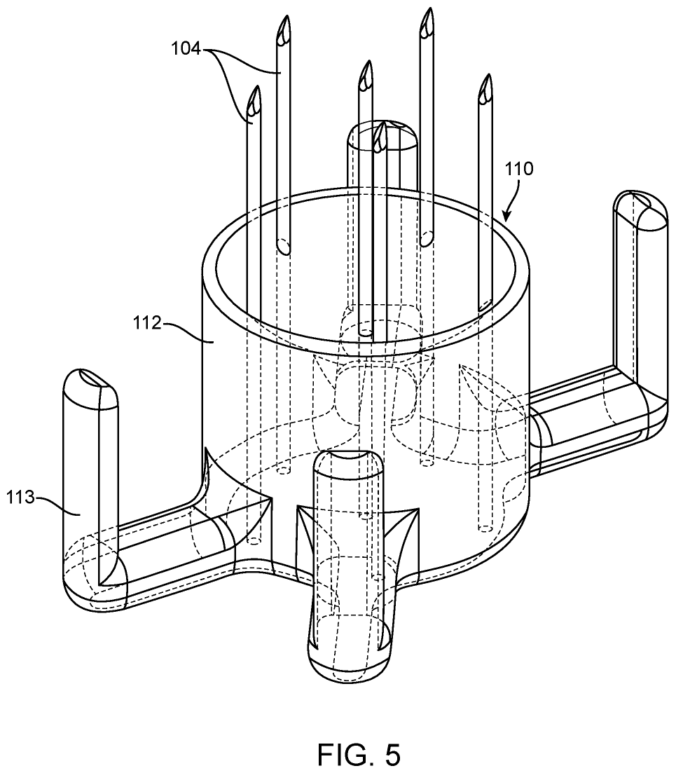

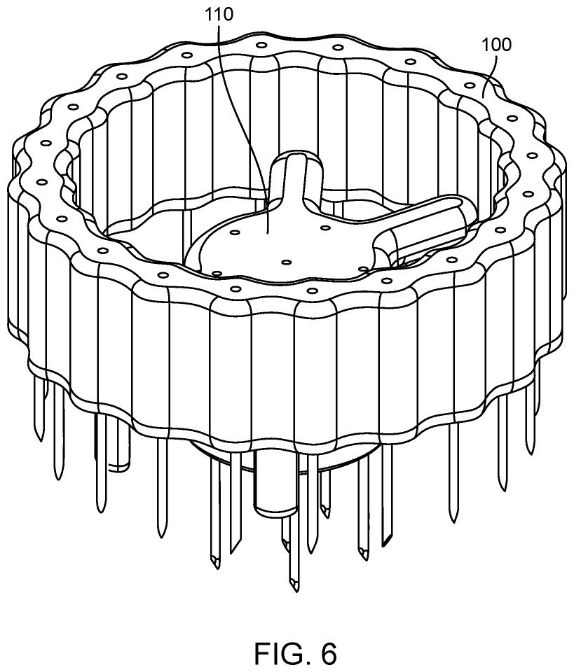

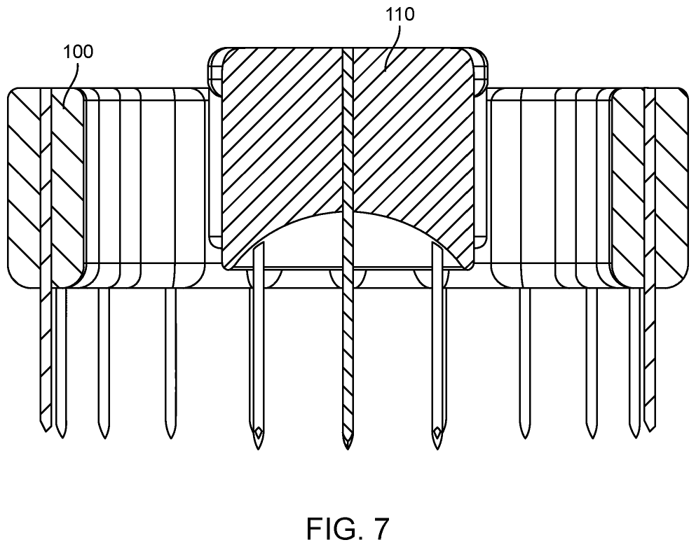

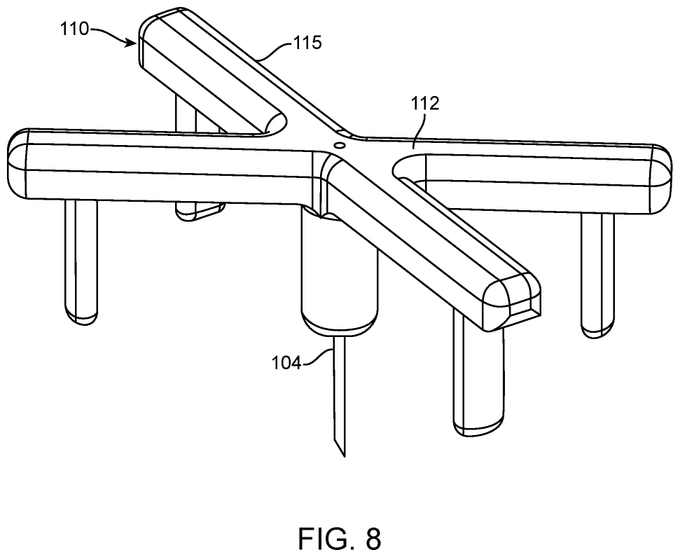

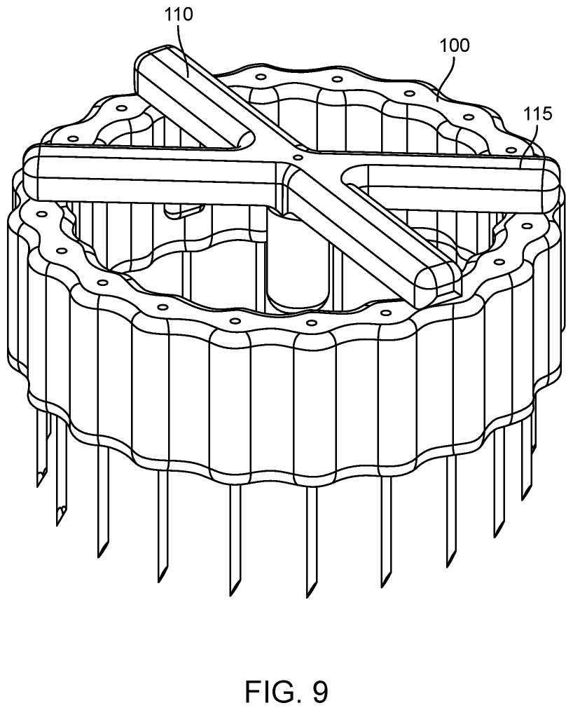

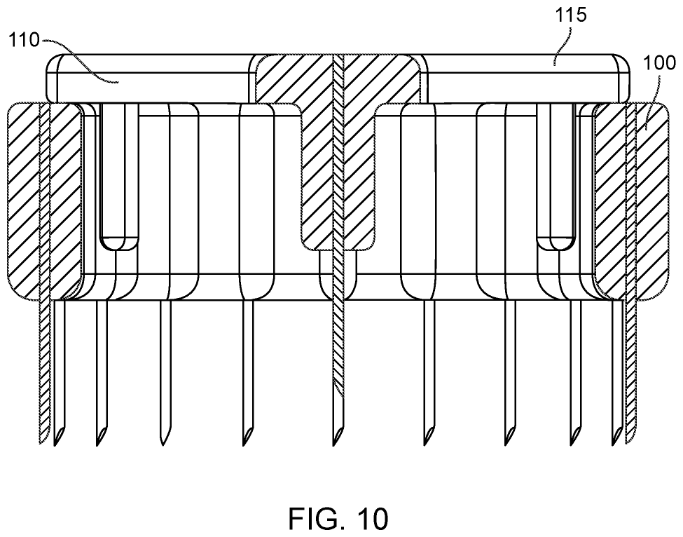

In one embodiment, the Ring Needle Assembly (100) is configured to mate with a Needle Hub Assembly (110), as shown in the perspective view of FIG. 5. The Needle Hub may slide, snap, ratchet, or otherwise be attachable to the Ring Needle Assembly. The Needle Hub Assembly and the Ring Needle Assembly may have features which limit their motion relative to one another. It may be desirable to connect the Needle Hub Assembly to the Ring Needle Assembly through the use of magnets or other methods. In another embodiment the Needle Hub is integrated together with the Ring Needle Assembly in one piece or permanently joined together. Similar to the Ring Needle Assembly, the Needle Hub Assembly has one or more needles (104) projecting beyond the surface for penetrating through any wound dressings, the graft, and the recipient site dermis. The skin facing surface of the Needle Hub may be flat. However, in some cases it is desirable for it to be a concave surface such that the margins of the Needle Hub are closer to the surface of the body and the center is raised. If only one needle is present, it is ideally centered in the Needle Hub and may have a longer length than the Ring Needle Assembly needles to account for potentially thicker graft material in the center of a graft while still allowing a length of the needle to penetrate into the recipient site dermis. One embodiment of the Needle Hub Assembly includes a central needle with a ring of multiple needles arranged concentrically around it. The Needle Hub may include one or more spacing members (113) which extend out radially from the Needle Hub, as shown in FIG. 5, to facilitate positioning of the needles when inserted within the Ring Needle Assembly such that the spacing members (113) may slide along an inner surface of the Ring Needle Assembly to help guide the needles along the Ring Needle Assembly (100), as shown in FIGS. 6 and 7. Alternatively, the central needle may be omitted and the Needle Hub may have a circular ring of needles. In another embodiment, the needles in the Needle Hub may be distributed in other patterns. In some cases, the face of the needle hub may have a layer of foam or other compressible material (156). In yet another embodiment, the one or more spacing members (115) may extend radially from the needles (104), as illustrated in FIG. 8, at a distance which is equal to or greater than a diameter of the Ring Needle Assembly. In such a configuration, the spacing members (115) may abut the top surface of the Ring Needle Assembly to prevent the further insertion of the needles (104) into the underlying tissue, thereby functioning as a limiting or stop feature which ensures the insertion depth into the tissue, as shown in FIGS. 9 and 10.

Needles (104) may be formed as part of the hub (112) and ring (102), for example molded from a very hard and stiff plastic or formed from metal. Needles may be separate elements that are insert molded, pressed into a separately produced ring or hub, glued or otherwise adhered into the ring or hub, welded to the ring or hub, snapped into the ring or hub, etc. In some cases, the needles may be produced from round wire which is cut to length and sharpened by grinding or similar processes. Other needles may be made by cutting and grinding sheet metal or wire of a different profile other than round. Needles may be machined. The needles may be coated over at least part of their length with silicone oil or other lubricant to reduce friction during insertion into tissue. The needles may be straight or may have one or more bends outside of the region which enters the tissue. Specifically, the bend or bends may be arranged to resist any tendency of the needle to pull out from the body of the ring or hub. Other geometry such as grooves, knurling, heading, or surface roughening may be utilized on the needle in a similar fashion. In some cases, the needle may be produced in a ‘U’ shape with both free ends sharpened (120).

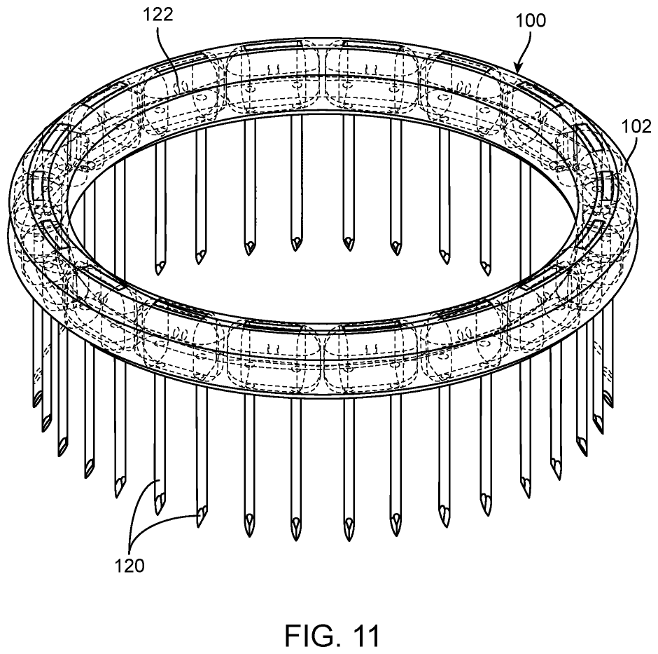

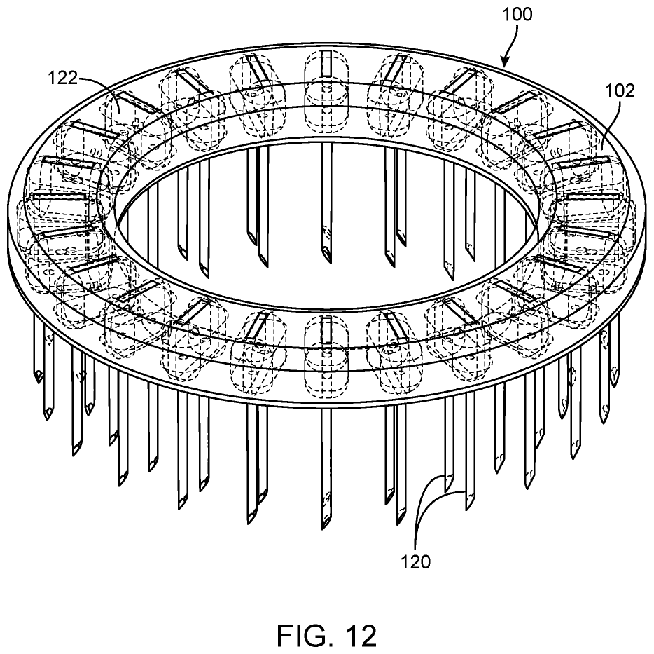

Overmolding of the needles may be conducted in one or more steps. For example, individual needles can be overmolded (122) and then groups of two or more overmolded needles could be overmolded again to form into a ring (102) or other shape. A similar process can be used for ‘U’ shaped needles as shown in FIG. 11 and FIG. 12.

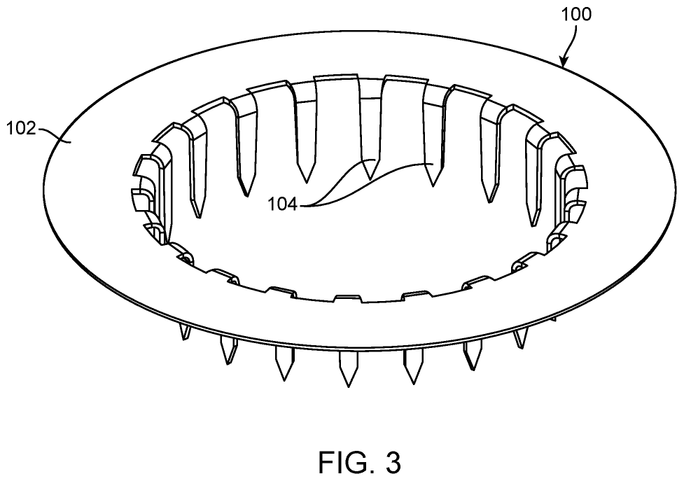

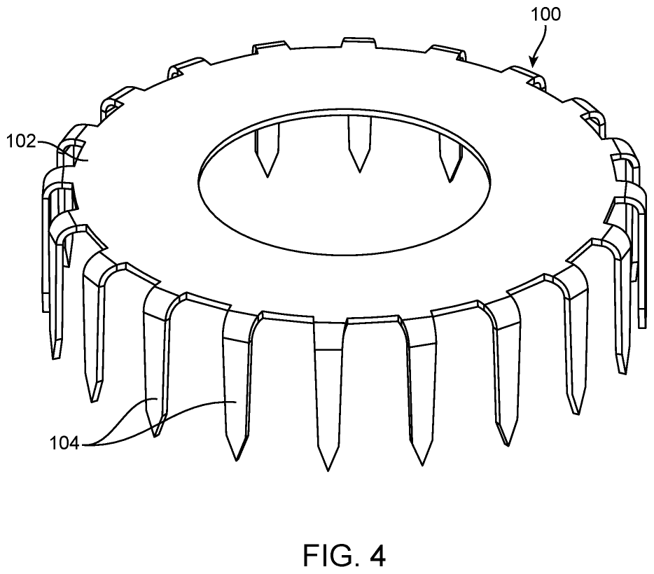

The needles may be produced from sheet metal with the needles remaining attached to the web of metal forming an annular ring with the needles arranged in a circular fashion to penetrate the tissue as shown in FIG. 3 and FIG. 4. In some cases, needles may be arranged on both the internal and external edges of such an annular ring. Such an annular ring may be overmolded with plastic as described above or may be permanently or removably attached to a larger piece of material for ease of handling or to prevent sharp edges of the sheet metal from being exposed inadvertently to the user.

In some cases, a detachable cap may be overmolded over each needle together with the body of the ring or hub. Alternatively, this may be added in a secondary step through overmolding or inserting the needle or needles into a resilient material such as silicone or other rubber. Such a cap may protect the needle tip from damage, protect the packaging from damage, protect users from inadvertent needle sticks during handling, and act as a sterile barrier. Such a cap may also serve as a platform which can be moistened to safely store the graft during surgery while awaiting transplantation to the recipient site.

For judging future position of the transplanted nipple-areolar complex, a Decal may be provided. The Decal may have a light adhesive to allow for easy repositioning on the new breast skin.

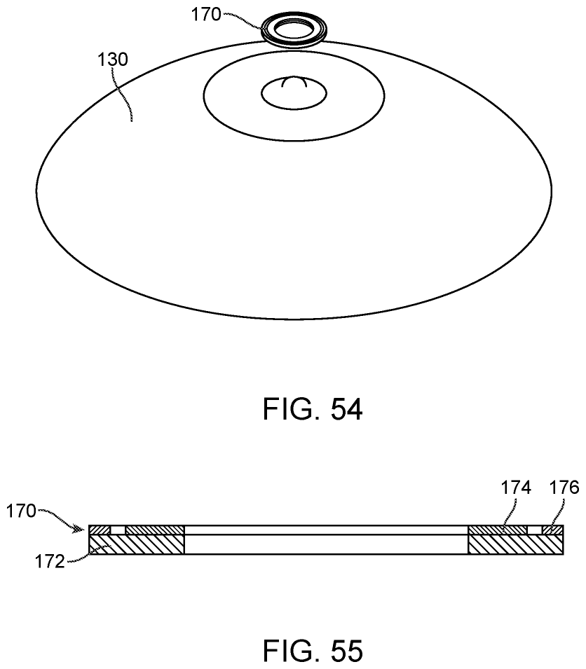

The Areola Transfer Patch (ATP) (132), Nipple Transfer Patch (NTP) (162), and Recipient Site Template (RST) (142) are comprised of one or more layers of material including a skin adhesive. Layers may include absorbent material such as foam, hydrocolloids, silicone, plastics, or other materials commonly used for surgical and wound care dressings. In some cases, these components may include one or more layers of a softer material such as hydrocolloid or absorbent foam and a stiffer and stiffer (PET or similar) support layer. The support layer may have a hole or series of holes to allow penetration of needles through the softer layer and tissues without needing to penetrate the stiff plastic layer. The example shown is an annular ring gap for needle penetration but individual holes for each needle are also foreseen (FIG. 53 and FIG. 54).

For pushing the needles through the graft, it is preferable to have a firm base to support the graft but which allows the needles to penetrate the base material. While this may be accomplished on the recipient site, the underlying tissue (such as adipose tissue) may not be firm enough to adequately support the graft during attempted needle penetration. In some embodiments, a prep tray may be provided which includes an appropriate material to support the graft during needle insertion. In addition, the material or a layer of prep tray may consist of a porous foam or other material such as gauze which can be wet with a physiologic or near physiologic crystalloid solution in order to maintain the moisture level of the grafts while awaiting the appropriate time for placement on the recipient site.

The prep tray described above may be configured in such a way that the foam or resilient material protects the needles of the hub and ring during transportation. This material may be in a tray with a removable lid with the needles of the hub and ring inserted in the material for sterilization, transportation, and/or storage.

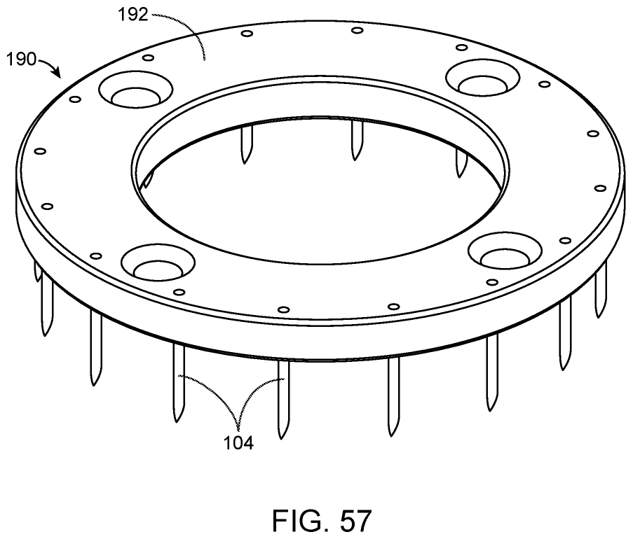

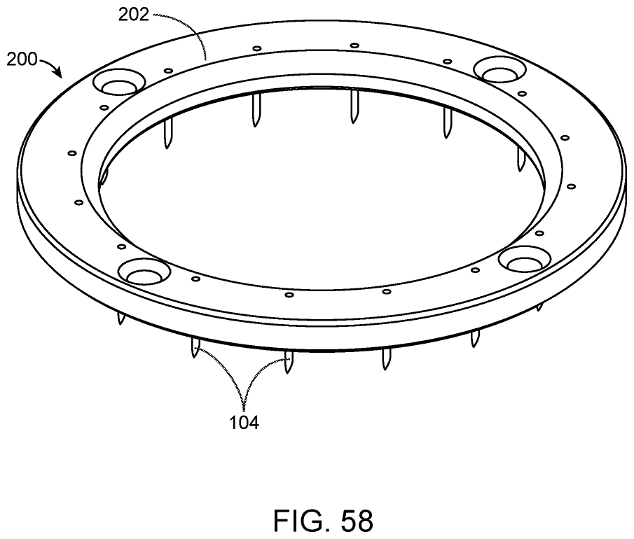

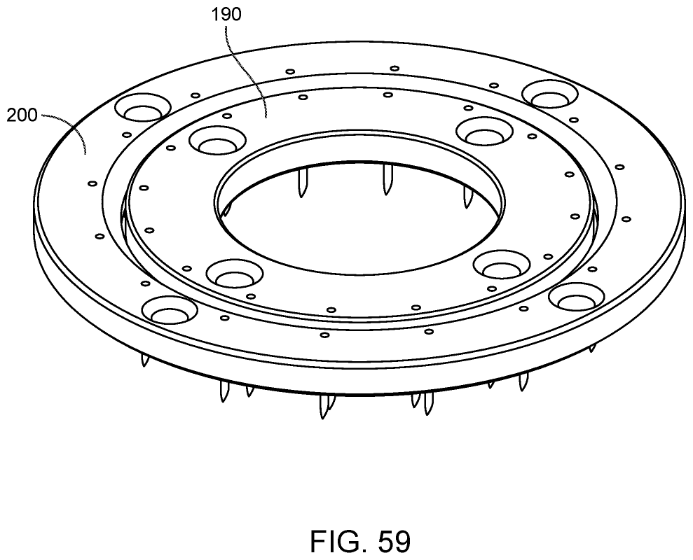

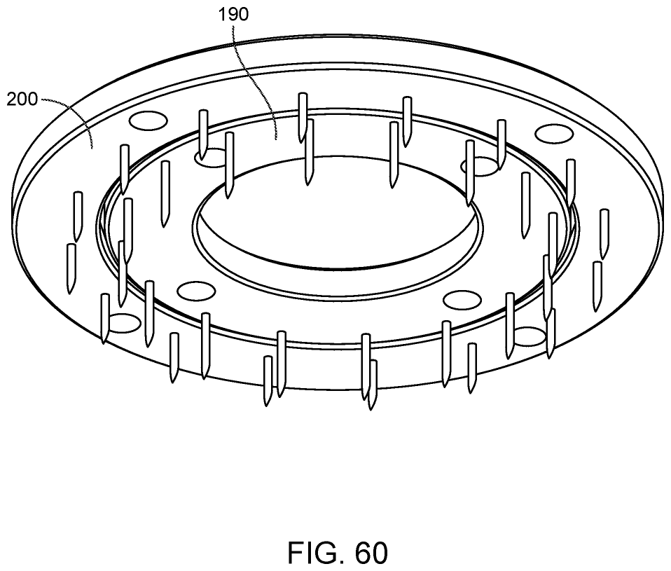

Another embodiment utilizes a first support structure, platform, ring (102) or disk with multiple needles (104) welded or otherwise attached to the ring with the needles configured to penetrate through the tissue close to the margin of the areola graft as shown in FIG. 57. The ring may be circular, oval, or any desired shape. Preferably the ring has a center hole to accommodate the nipple projection and this hole may be circular, oval, or other desired shape. The needles can be solid or canulated (hollow) with points configured to penetrate through tissue and of a length sufficient to extend through the harvested graph and into the underlying dermis or other tissues at the recipient site. A second ring or disk (as shown in FIG. 58), which has a hole configured to fit around the first ring as shown in FIG. 59 and FIG. 60 such that the support structures, platforms or rings (190, 200) are configured in a concentric arrangement within a common plane where the needles from both rings (190, 200) may be adjacent to one another so that they extend in the same direction for engagement with the underlying tissue, also with multiple needles configured to penetrate through the tissue close to the margin of the recipient site may also be used. Similar to the first ring, this second ring may be circular, oval, or any desired shape. The needles may be attached to the rings through welding, soldering, brazing, gluing, or similar methods for attaching similar or dissimilar materials. In one embodiment, the rings are thin so as to provide a minimal projection above the skin after the needles are pushed into the tissue. The thickness of the rings is preferably less than, e.g., 10 mm, and ideally more than, e.g., 3 mm. The thinner ring also allows for better access to the tissue inside the second ring for cutting and de-epithelization. The rings can have holes in them (or other features such as projections) to allow for sutures, staples, or other securement devices securable to each of the rings (or to a single ring) for holding the rings to the tissue and/or to one another during the healing period. In some embodiments, these holes have chamfers or radiused edges to minimize the stress on a securement suture passing through the hole. The rings may also have one or more features which may allow for the rings to be secured to one another once deployed upon the tissue (or before deployment) which may keep or maintain an orientation or position of the rings relative to one another to prevent or inhibit inadvertent movement of the rings relative to one another. Additionally, it may be desirable in some cases to place a full or partial thickness bevel on the inner edge of the second ring to allow the user to guide a scalpel or other cutting instrument either vertically along the edge or angled along the bevel to cut the margin of the recipient site, the bevel additionally providing enhanced access for instruments to remove the epidermis during de-epithelization.

While needles are the ring-shaped structures are shown in the embodiment above, the use of concentric platforms may be used with any of the piercing embodiments and/or with any of the different platform embodiments described herein. Different features between different embodiments are expressly intended to be used in any number of combinations, as practicable.

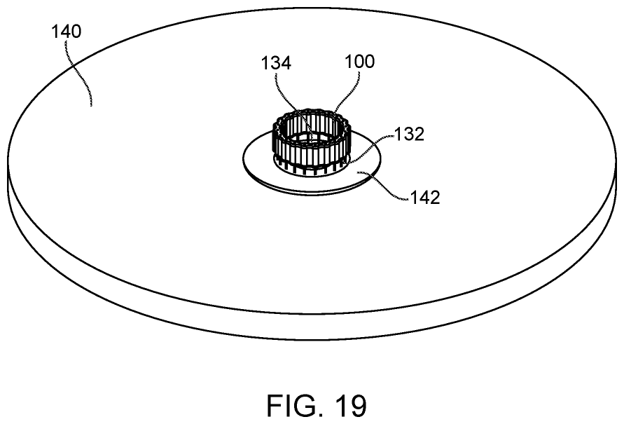

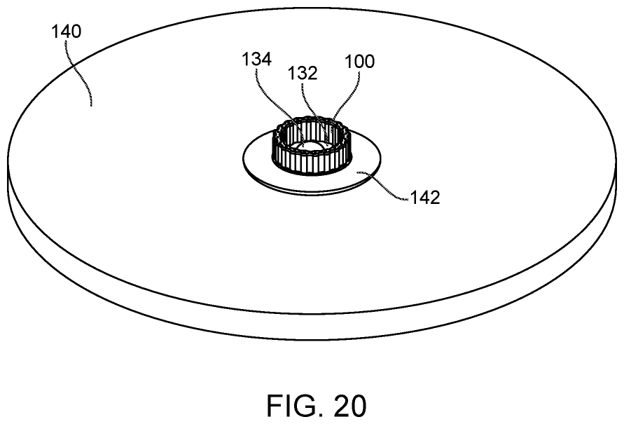

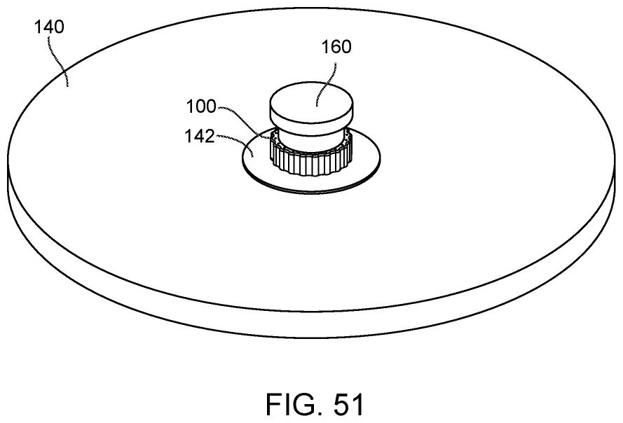

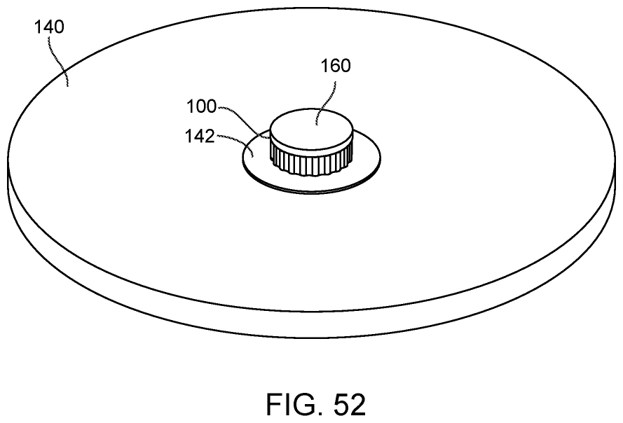

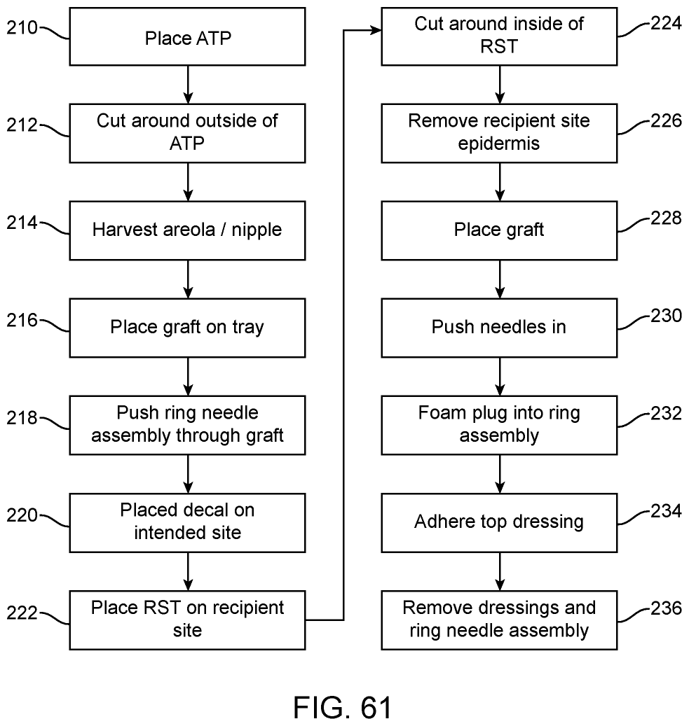

A method is outlined in the block diagram of FIG. 61, for grafting a section of tissue such as a nipple-areola complex. In one embodiment the method is used to transfer the nipple and areola in one piece. In this method, an adhesive bandage of the desired size and shape (Areola Transfer Patch, ATP) is placed (210) on the harvest site (FIG. 13). A scalpel, areola cutter, or other cutting device is used to cut the tissue to a shallow depth around the outer edge of the ATP (212). The areola and nipple are harvested (214) in one piece (FIG. 14). The harvested graft is set on a preparation tray (216), which is a moist platform which supports the graft while minimize the graft's tendency to dry. A Ring Needle Assembly such as described above is placed onto the graft (218) with the needles of the assembly at least partially penetrating through the graft (FIG. 15). A decal or other surrogate image of the nipple-areolar complex can be placed on the desired position for the graft recipient site (220). Use of a decal which can be repositioned is advantageous in providing a visual guide for selecting a visually appropriate location. An adhesive bandage (Recipient Site Template, RST) similar to the ATP can be placed on the recipient site (222) around the positioned decal (FIG. 16). The tissue can be cut around the inner edge of the RST using a scalpel, an areola cutter, or other cutting device (224). The epidermis is then removed from the recipient site (226) (FIG. 17). The Needle Ring with ATP and areola/nipple graft is placed on the recipient site (228) (FIGS. 18 and 19). The Ring Needle Assembly is pushed so that the needles are fully penetrating the tissues (230) (FIG. 20). A foam plug or other compressible material can then be placed into the center area of the Ring Needle Assembly (232) such that pressure can be applied by the final dressings against the graft and the chest wall (FIG. 51 and FIG. 52). A top dressing such as a thin adhesive bandage or other medical adhesive sheet is then adhered over the foam plug and the Ring Needle Assembly and adhered to the surrounding chest skin to hold the device in place (234). When appropriate (typically, e.g., 5-10 days later), the dressings and the Ring Needle Assembly are removed (236) (FIG. 53).

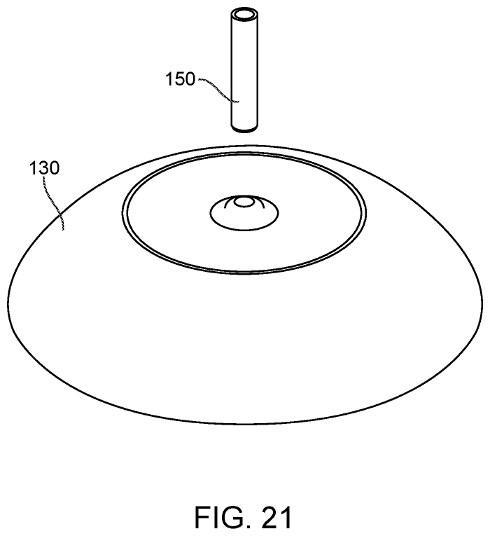

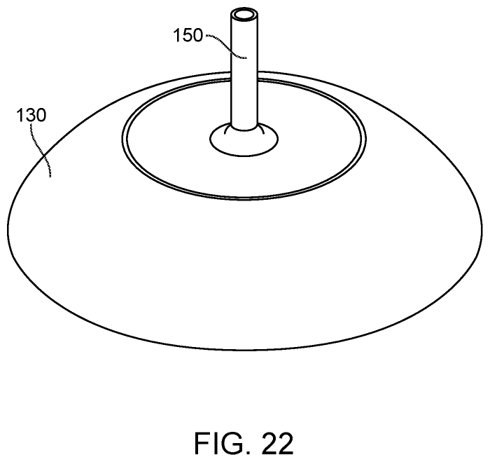

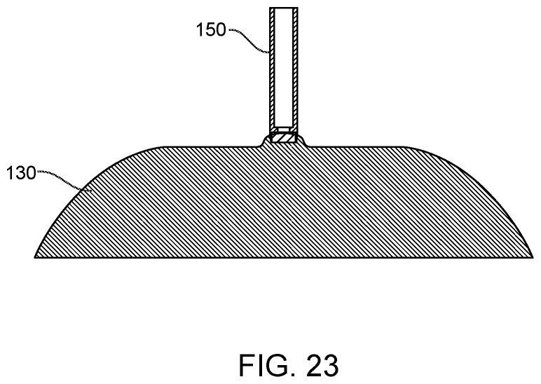

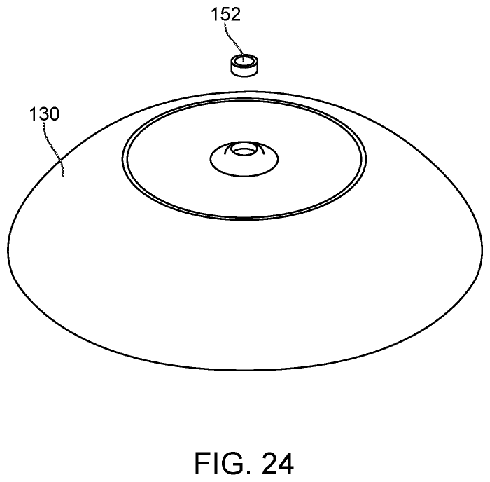

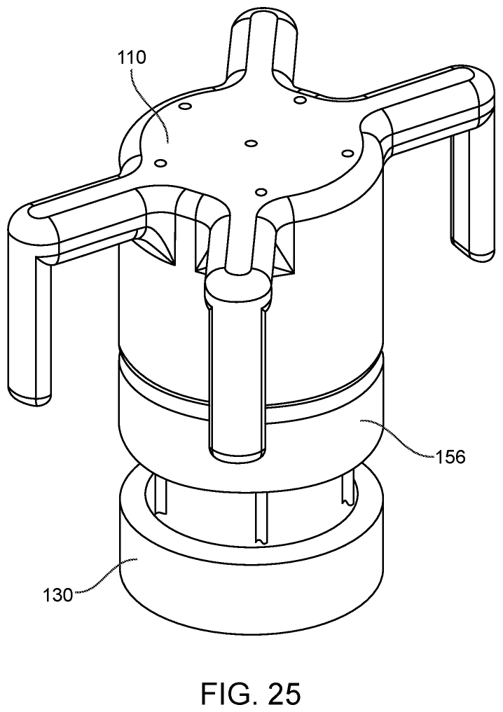

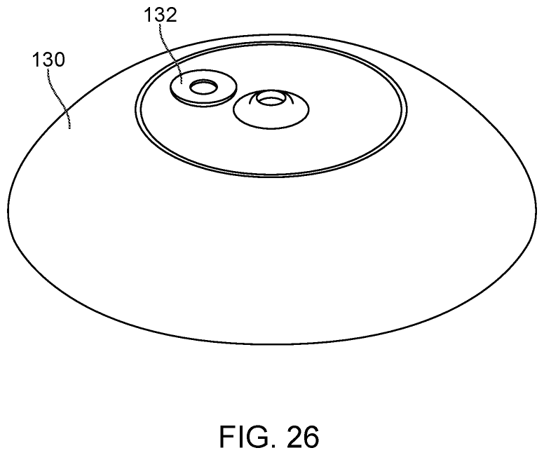

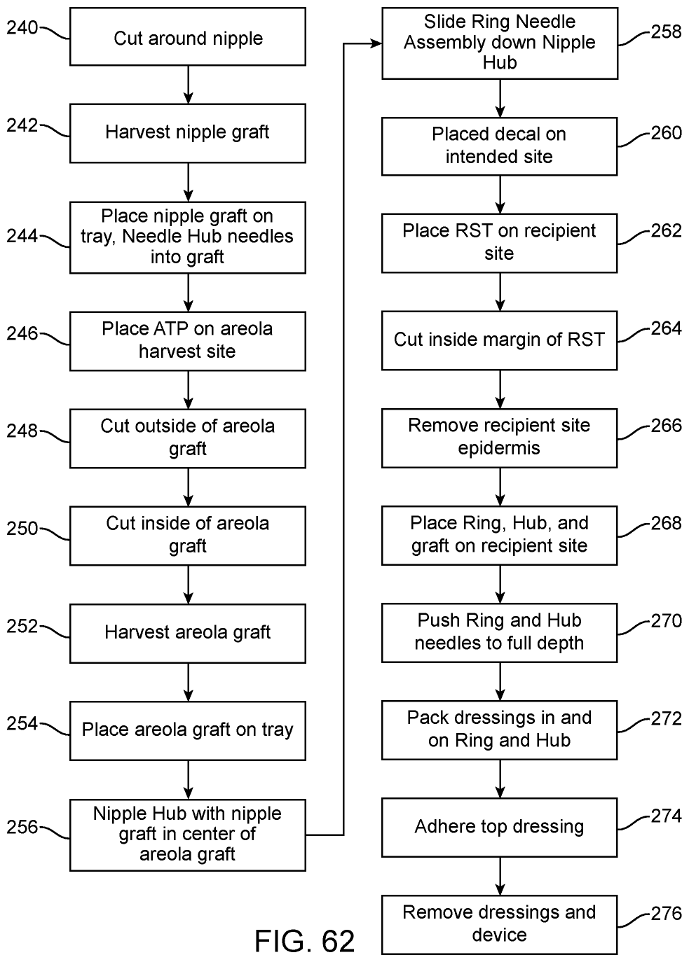

An alternate method is presented in the flowchart of FIG. 62 for grafting tissue in two pieces such as a nipple and areola harvested separately, for example, where nipple anatomy and/or texture makes it less than ideal as a source for areolar skin. The method utilizes a Ring Needle Assembly (100) and a Needle Hub Assembly (110). A Nipple Cutter (150) (ideally, e.g., 5-10 mm diameter) is used to cut the circumference of a portion of the nipple for grafting (240). The cutter is preferably sharp and able to cut to desired depth in tissue (e.g., 2-8 mm). The cutter may be smooth, serrated, or not sharp enough to cut but to leave ink or a blanching impression in skin to mark the desired incision (FIG. 21, FIG. 22, and FIG. 23) for cutting with a different instrument. The nipple graft is then harvested from the underlying tissue (244) (FIG. 24) and the harvested nipple graft is placed on a moistened platform (prep tray) and the needles of the Needle Hub assembly are inserted into the graft (FIG. 25). The Areola Transfer Patch (ATP, previously described) is placed on the skin in the desired area for harvesting the areola graft (246) (FIG. 26).

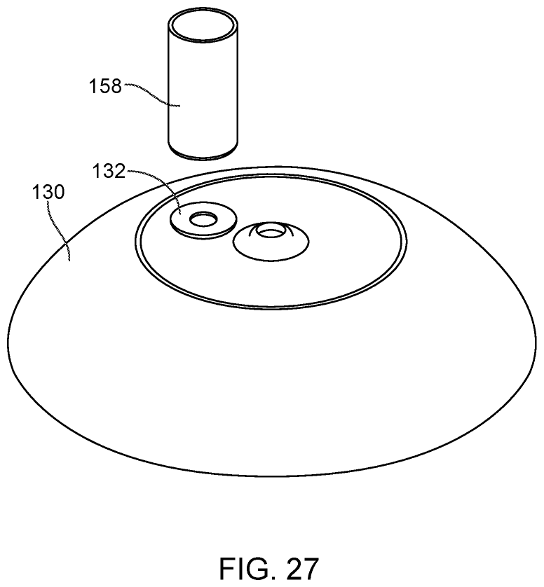

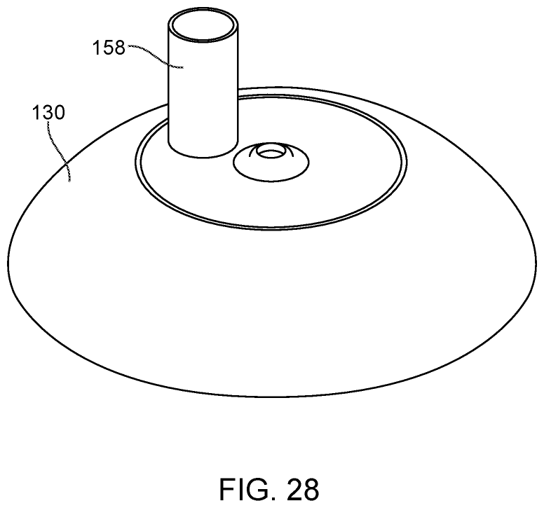

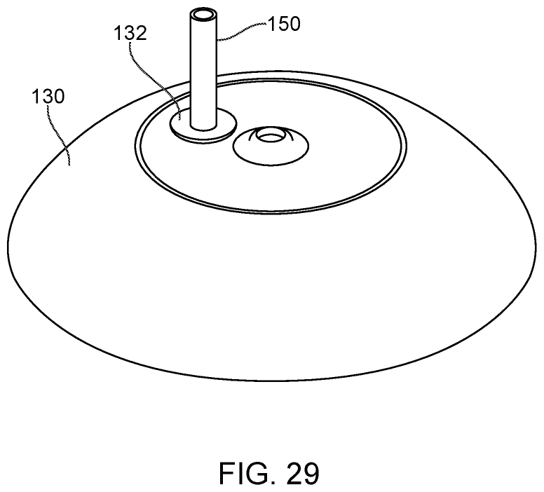

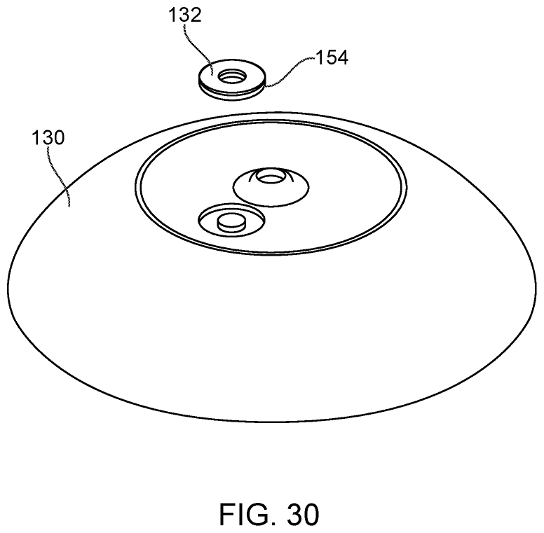

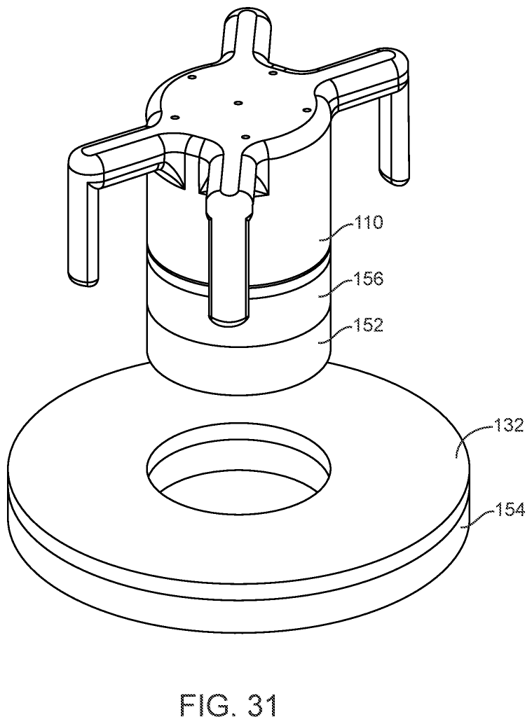

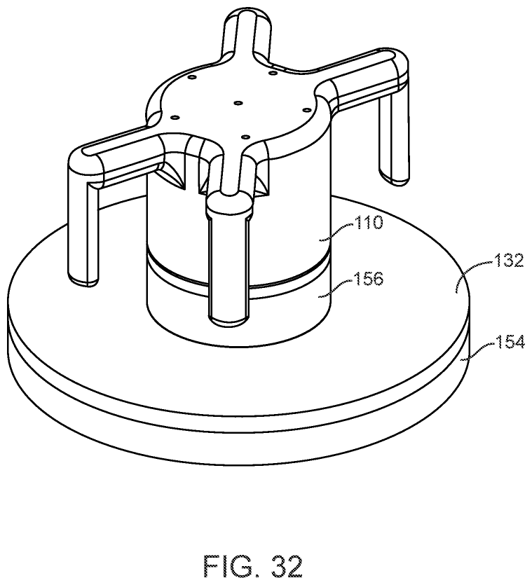

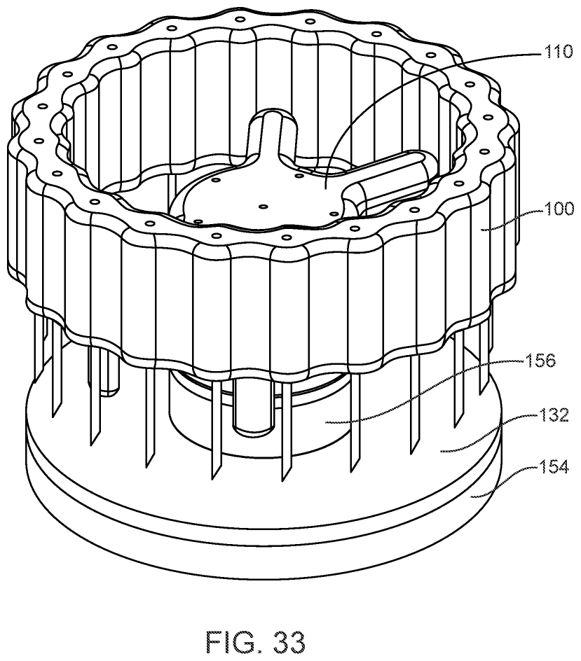

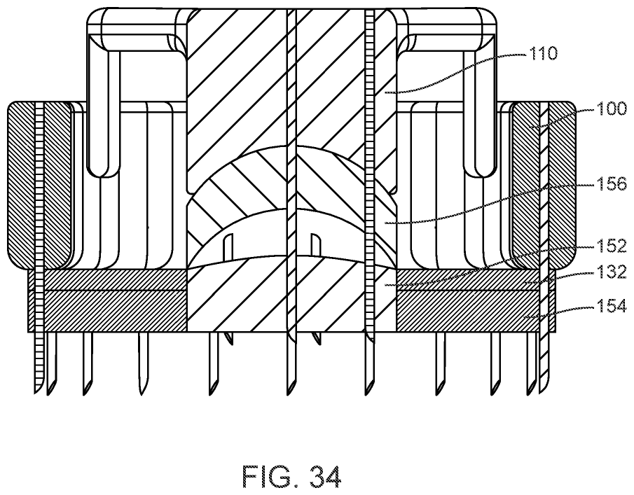

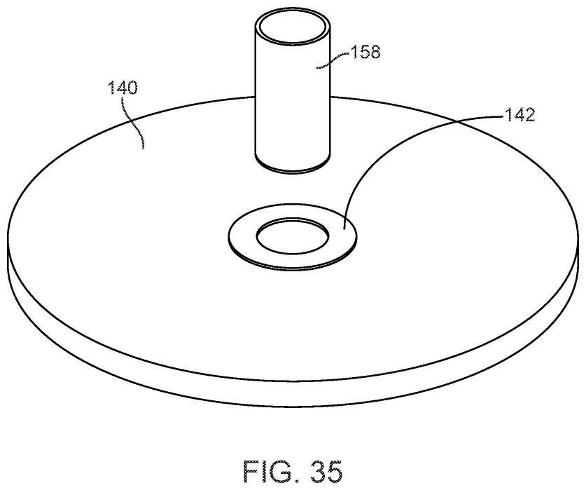

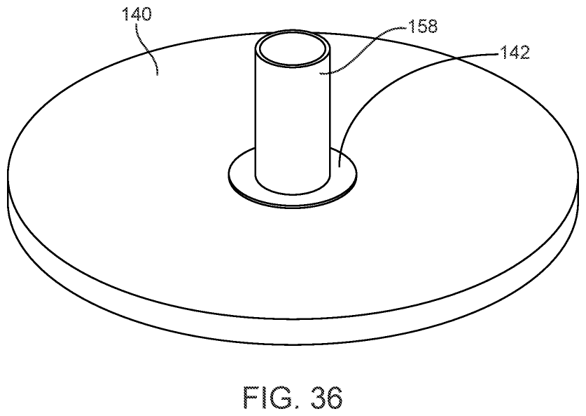

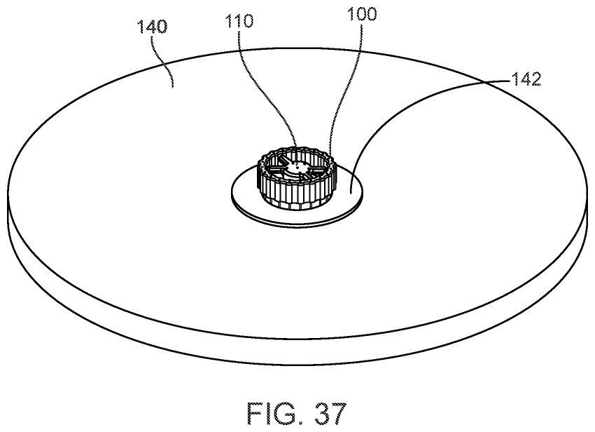

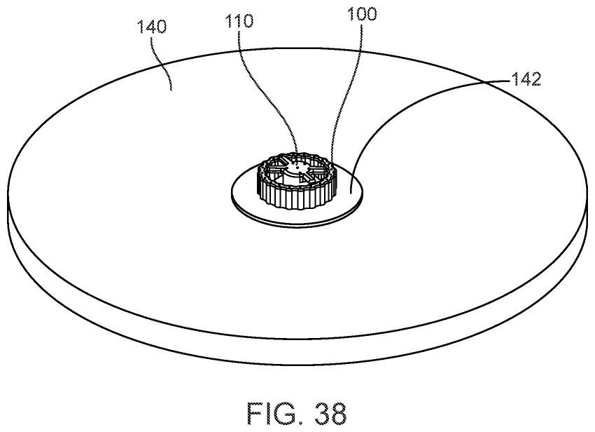

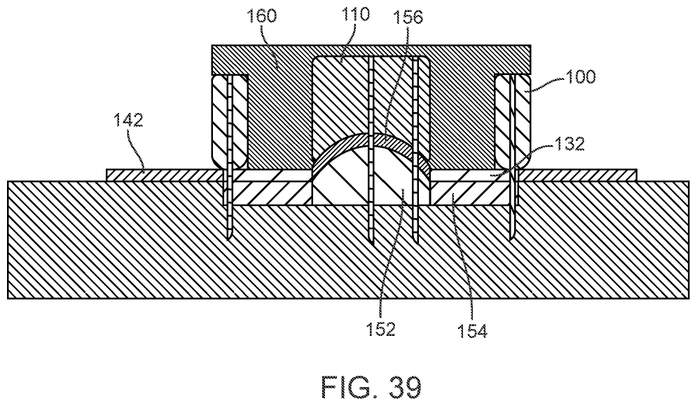

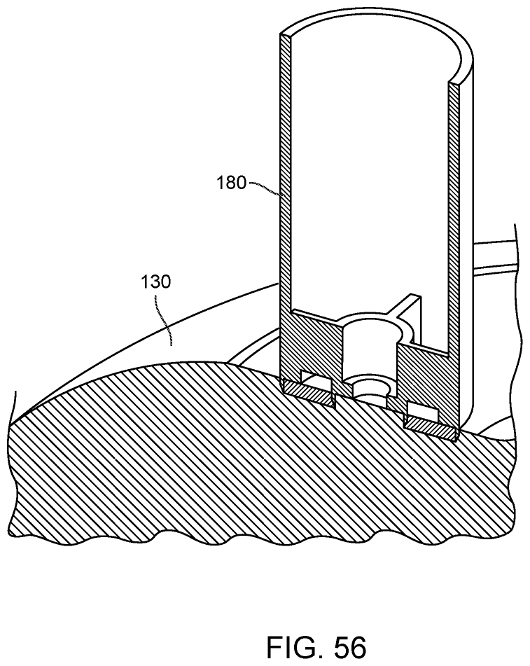

An Areola Cutter (158), similar to the Nipple Cutter but of a larger diameter (e.g., 22-25 mm or similar, or oval of similar size) can then be used to cut around the outside margin of ATP (248) (FIG. 27 and FIG. 28). Alternatively, a dual ring Areola Cutter can be used to cut inner and outer margins of the areolar graft simultaneously (FIG. 56). The inner margin of the areola graft is then cut using the Nipple Cutter (creating annular areola graft) (250) (FIG. 29). This step is skipped if dual ring Areola Cutter is used to cut both margins simultaneously. Alternatively, the inner margin of areola graft may be cut on the prep tray after harvesting complete piece of areola. The areola graft is then harvested by separating it from the underlying tissues (252) (FIG. 30). The harvested areola is placed on a moist prep tray (254). The nipple graft with Needle Hub that was previously harvested is placed in the areola graft so that the nipple graft is located in the hole in the center of the areola graft (256) (FIG. 31 and FIG. 32). The Ring Needle Assembly is slid down the Needle Hub until the needles have penetrated the outer margin of the areola (258) (FIG. 33 and FIG. 34). A decal or other surrogate image of the nipple-areolar complex can be placed on the desired position for the graft recipient (260). Use of a decal which can be repositioned is advantageous in providing a visual guide for selecting a visually appropriate location. The Recipient Site Template (RST, previously described) is then placed around the desired recipient site (262) (FIG. 16). The Areola Cutter can then be utilized to cut partial thickness of skin at the inner margin of the RST (264) (FIG. 35 and FIG. 36). The epidermis above the recipient site is removed and discarded (266) (FIG. 17). The needle Assembly, Needle Hub Assembly, and previously harvested graft are placed on the recipient site (268) (FIG. 37). Once the graft is in the desired position, the penetrating elements of the Ring and Hub can be pushed to full depth in the tissues, securing the graft in place (270) (FIG. 38). The space between the Hub and the Ring are then packed with cotton soaked in petrolatum (or other soft, compressible material such as foam with a surface that will tend to not stick to the wound/skin). Additional dry cotton or soft material is placed over top of hub such that it is proud of the Hub surface and the top of the Ring (272) (FIG. 39, FIG. 51, and FIG. 52). An adhesive medical bandage such as Tegaderm or similar is then adhered over cotton and device to hold the device and grafts against the chest (274). When appropriate (typically, e.g., 5-10 days later), device and dressings are removed (276) (FIG. 53).

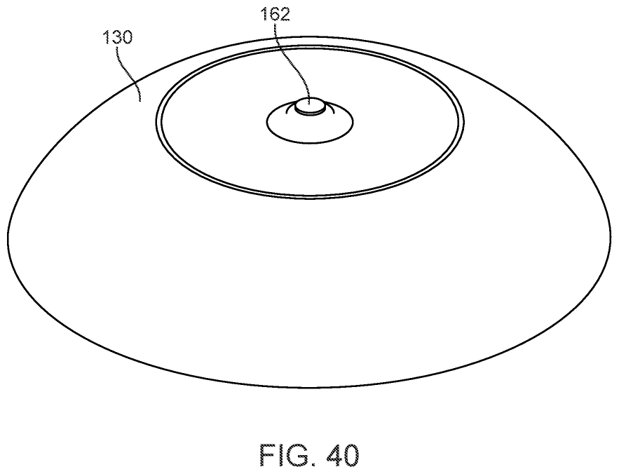

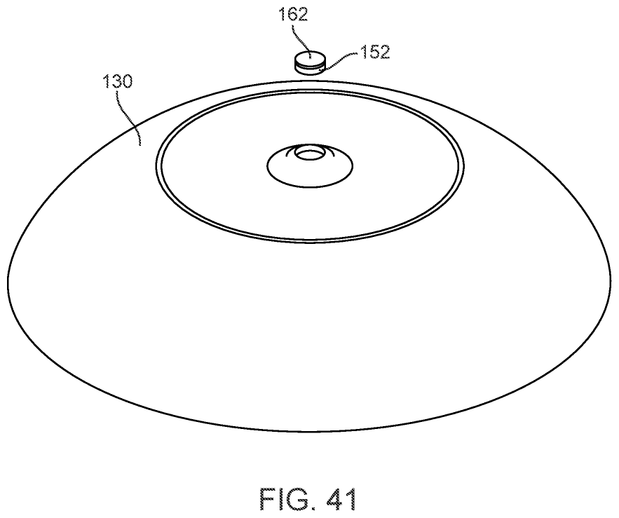

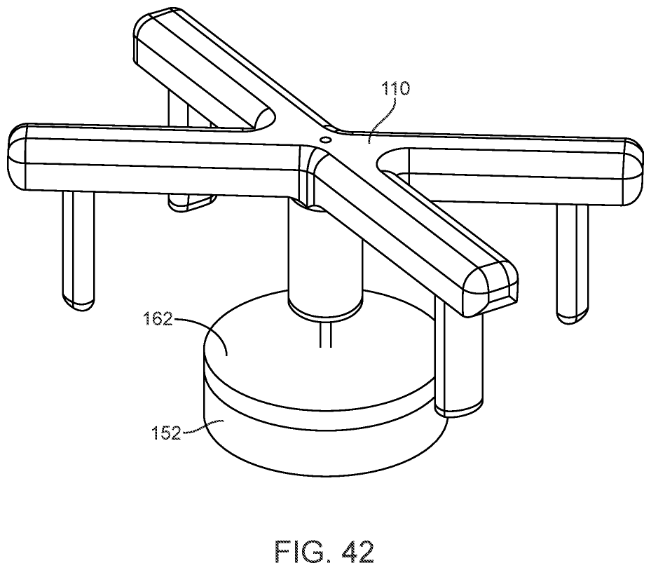

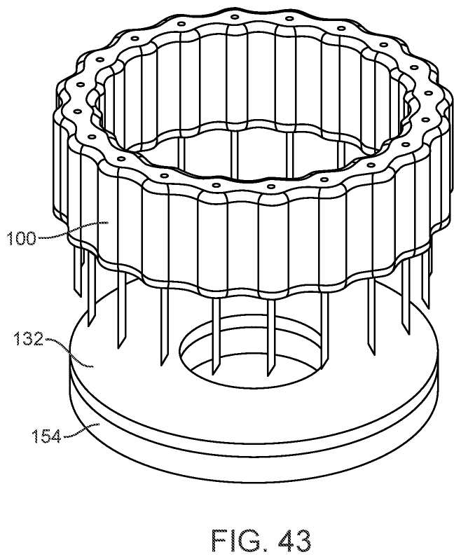

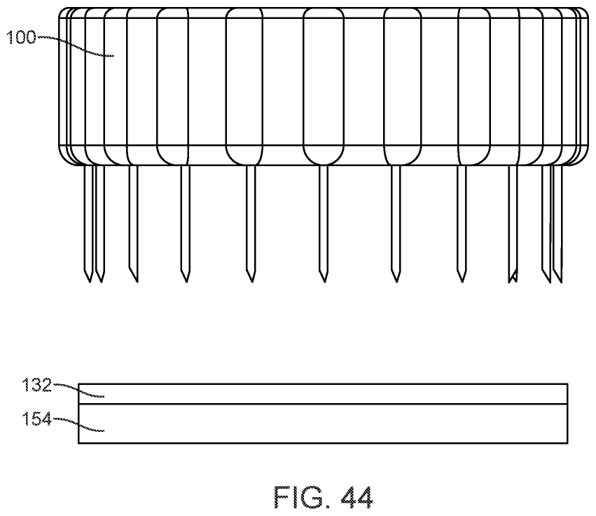

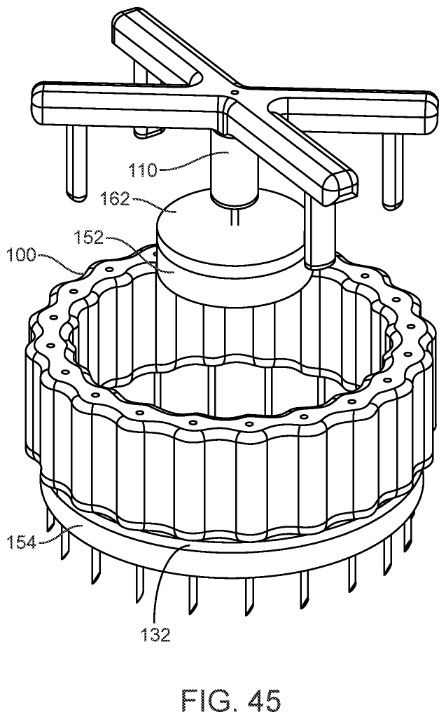

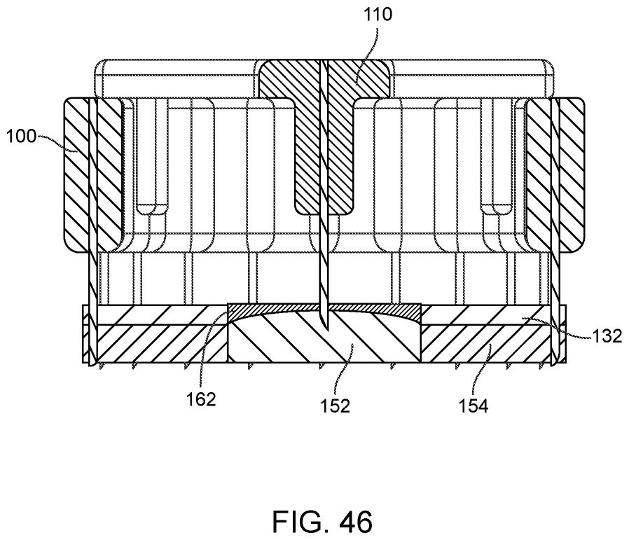

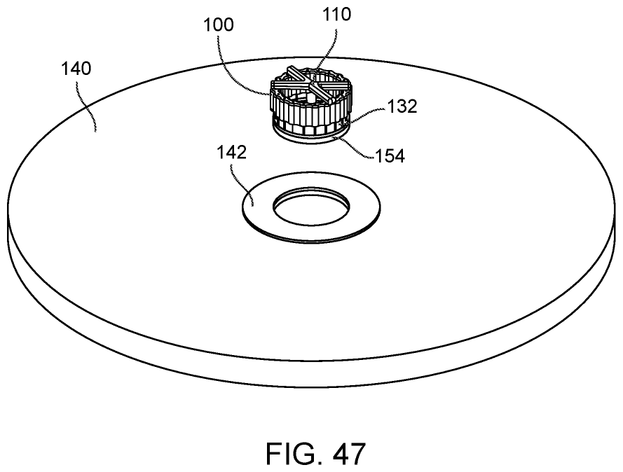

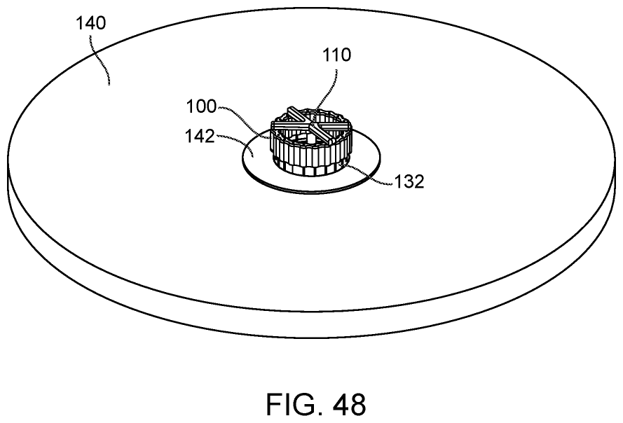

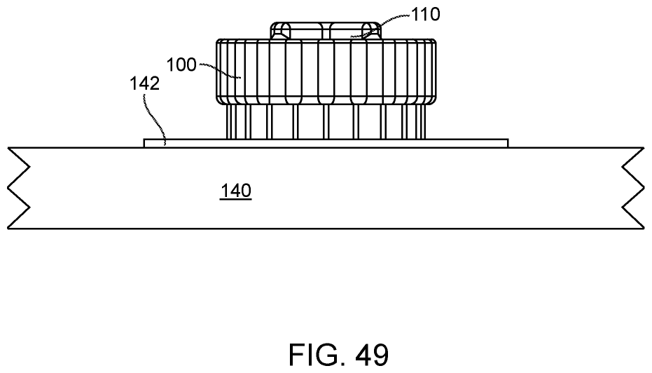

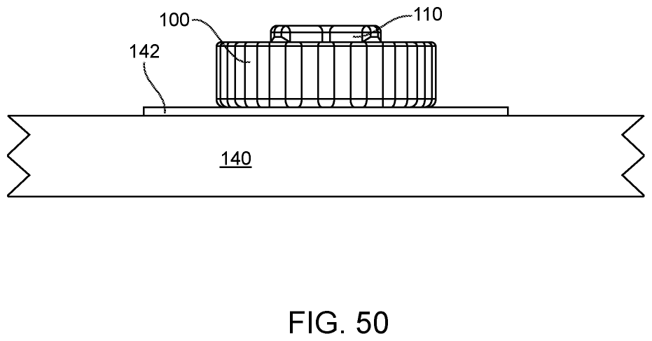

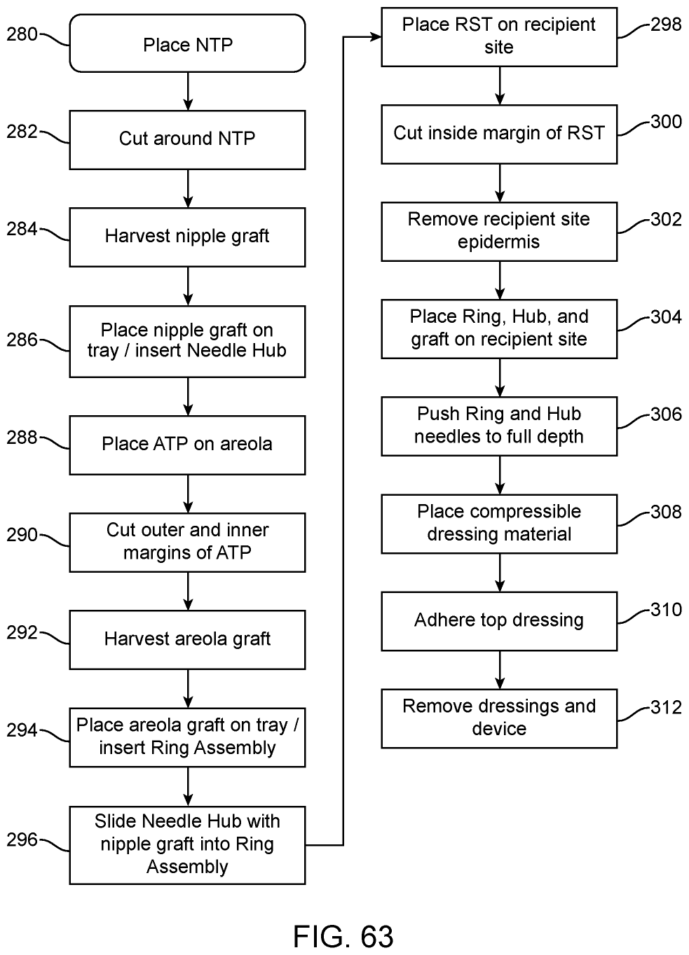

The flowchart of FIG. 63 shows an alternate method for grafting tissue in two pieces such as a nipple and areola harvested separately. The method utilizes a Ring Needle Assembly (100) and a Needle Hub Assembly (110) and transfer stickers for both the areola and nipple (ATP (132), NTP (162). A Nipple Transfer Patch (NTP) is placed on the harvest site (280) (FIG. 40). This step is optional. If omitted, the nipple is harvested and handled without the use of a transfer patch. A scalpel or other cutting device is used to cut the tissue around the outer margin of the NTP (282). The nipple graft is harvested by separating the graft from the underlying tissue (284) (FIG. 41). The needles of the Needle Hub assembly are inserted through the nipple graft after the graft is placed on a moist prep tray (286) (FIG. 42). Subsequently an Areola Transfer Patch (ATP) is adhered to the areola in the area that is desired to be harvested (288) (FIG. 26). The outer and inner margins of the tissue below the ATP can then be cut (290). By cutting underneath the intended areolar graft between the inner and outer margins, the graft is harvested (292) (FIG. 30). The harvested areolar graft can be placed on a moist prep tray and the Ring Needle Assembly needles pushed into the graft (294) (FIG. 43 and FIG. 44). The Needle Hub with nipple graft are slid into Ring Needle Assembly so that grafts are concentric with needles at least partially penetrating the grafts (296) (FIG. 45 and FIG. 46). A Recipient Site Template (RST) is then placed on the intended recipient site (298) (FIG. 16). Using a scalpel or other appropriate device, the skin is cut under the inner margin of the RST (300). The epidermis in the recipient site is then removed by separating it from the underlying tissue (302) (FIG. 17). The Ring, Hub, and graft are positioned on the prepared recipient site (304) (FIG. 47). After confirming the positioning of the graft within the RST is correct, the device is pushed so that the needles of the Ring and Hub assemblies are penetrating the tissue to full depth (306) (FIG. 48, FIG. 49, and FIG. 50). Soft, compressible material such as foam with a surface that will tend to not stick to the wound/skin is then placed. Preferably this compressible material is provided with projections that fit in space between Ring Needle Assembly and Needle Hub (308) (FIG. 51 and FIG. 52). Optionally, additional dry cotton or soft material may be placed over the top of the Hub such that it is proud of the Hub surface and the top of the Ring Needle Assembly. A top dressing (not shown, described elsewhere in this document) is placed over the device and graft and adhered to the chest (310). When appropriate (typically, e.g., 5-10 days later) device and dressings are removed (312) (FIG. 53).

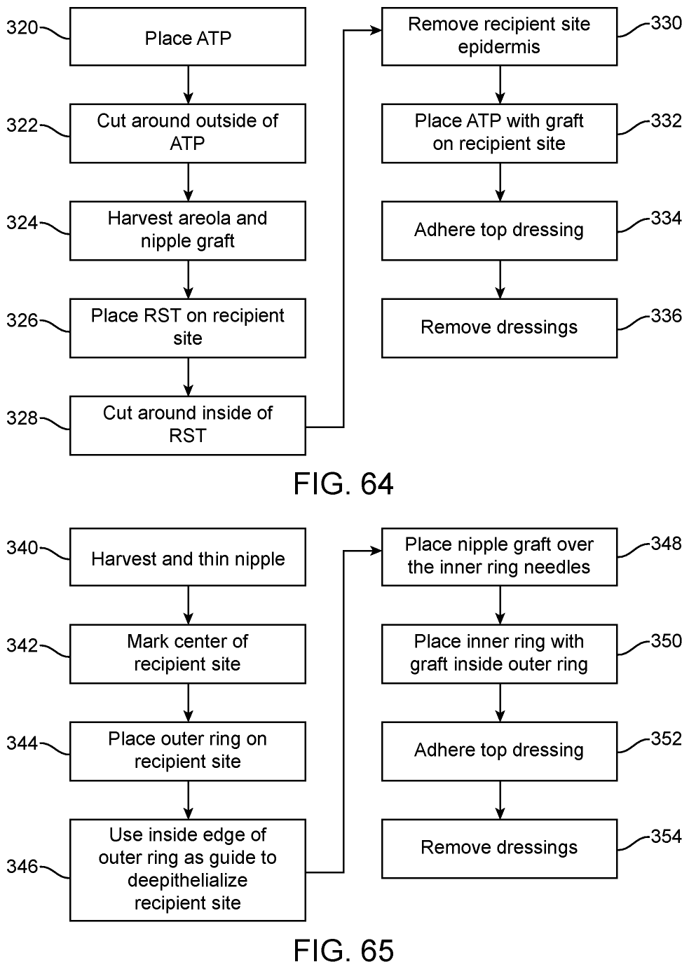

FIG. 64 is a block diagram of another alternate method (FIG. 64) for nipple harvest and grafting utilizing a transfer sticker for the areola and the nipple in one piece. This method does not utilize a Ring Needle Assembly or Needle Hub Assembly. An Areola Transfer Patch (ATP) is placed on the graft harvest site (320) (FIG. 13). The skin under the outer margin of the ATP is cut (322). This may be done using a scalpel, an Areola Cutter, or other cutting device. The areola and nipple are harvested together by cutting underneath them to separate them from the underlying tissue (324) (FIG. 14). A Recipient Site Template (RST) is placed on the desired recipient site (326) (FIG. 16). The skin under the inner margin of the RST is then cut (328). This may be done using a scalpel, an Areola Cutter, or other cutting device. Next the recipient site is prepared by cutting underneath the epidermis and removing it (330) (FIG. 17). The ATP with graft is placed on recipient site (332) (FIG. 18 and FIG. 19). Subsequently, a dressing is placed over the graft, ATP, and RST and adhered to the chest (334). An intervening compressive material may be inserted between the top dressing and underlying grafts if desired. When appropriate (typically, e.g., 5-10 days later), dressings and device are removed (336) (FIG. 53).

A flowchart of a different method of nipple graft preparation and anchoring is shown in FIG. 65. In this embodiment, the nipple is harvested and thinned by the surgical team in the usual manner such as described elsewhere in this document (340) (e.g., using the embodiment of the first and second platforms (190, 200) described herein with respect to FIGS. 57-60). When the chest site is ready for grafting, the surgeon determines and marks the center of the recipient site (342). The outer ring with needles is placed into the recipient site such that the center of the ring aligns with the center mark from the previous step (344). This step not only defines the area of deepithelialization, but also prevents the outer skin of the recipient site from pulling away from the recipient site after deepithelialization. The upper edge of the inner diameter of the outer ring may be beveled. Utilizing the bevel on the inner diameter of the outer ring to guide a scalpel blade, the recipient site is de-epithelialized (346). The thinned nipple is then placed over the inner ring needles (348), optionally utilizing slotted “Russian” forceps or similar instruments to facilitate the maneuver. The inner ring is then placed inside of the already positioned outer ring (350). The rings are pushed so that needles are fully seated in the tissue. An adhesive bandage or other dressing is adhered to the skin to hold rings (and graft) in place (352). Optionally or additionally, a compressive binder may be applied around the torso over the bandages. When appropriate (typically, e.g., 5-10 days later), bandages and rings are removed (354) (FIG. 53).

In cases where the nipple graft has excess skin at the circumference after being placed over the inner ring needles, subsequent placement of the inner ring inside of the outer ring may lead to upward displacement and compression of the excess skin, thus facilitating its ultimate scab formation and separation later from the desired healing graft area. The steps outlined above need not be in the exact order described, or different methods may be partially combined as desired. Additionally, several options exist to anchor the system according to the surgical team's preferences. In some embodiments, holes may be present in the inner and/or outer rings which may be used to suture the rings to the underlying tissue. Also, similar to other embodiments described herein, a soft dressing of the surgical team's choice may be placed over the system, possibly held in position with an adhesive covering followed by a typical chest binder.

The applications of the devices and methods discussed above are not limited to transferring the nipple areolar complex but may include any number of further treatment applications. Moreover, such devices and methods may be applied to other treatment sites within the body. Modification of the above-described assemblies and methods for carrying out the invention, combinations between different variations as practicable, and variations of aspects of the invention that are obvious to those of skill in the art are intended to be within the scope of the claims.

What is claimed is:

1. A tissue transfer apparatus, comprising:
a first support structure having an elliptical or circular configuration sized to encircle a region of tissue;
a first plurality of piercing features projecting from a first side of the first support structure;
a second support structure having an elliptical or circular configuration sized to encircle the region of tissue and defining an opening which is further configured to encircle the first support structure; and
a second plurality of piercing features projecting from a first side of the second support structure such that the first and second piercing features are adjacent to one another.

2. The apparatus of claim 1 further comprising a securement mechanism attachable to the first support structure for securement to the region of tissue.

3. The apparatus of claim 2 wherein the securement mechanism is attachable to the second support structure for securement to the region of tissue.

4. The apparatus of claim 1 further comprising a transfer substrate configured to approximate a size of the region of tissue when positioned upon the region of tissue.

5. The apparatus of claim 4 wherein the transfer substrate comprises an adhesive substrate.

6. The apparatus of claim 1 wherein the first support structure and the second support structure are configured to encircle the region of tissue comprising a nipple-areolar complex.

7. The apparatus of claim 1 wherein the first support structure comprises a ring-shaped member.

8. The apparatus of claim 7 wherein the second support structure comprises a ring-shaped member.

9. The apparatus of claim 1 wherein the first support structure and the second support structure form a concentric arrangement within a common plane.

10. The apparatus of claim 1 wherein the first plurality of piercing features comprises needles.

11. The apparatus of claim 10 wherein the second plurality of piercing features comprises needles.

12. The apparatus of claim 1 wherein the first plurality of piercing features comprise flat needles positioned orthogonally from an inner or outer diameter of the first support structure.

13. The apparatus of claim 12 wherein the second plurality of piercing features comprise flat needles positioned orthogonally from an inner or outer diameter of the second support structure.

14. The apparatus of claim 1 further comprising a recipient site template configured to receive the first support structure or the second support structure when the recipient site template is positioned upon a recipient site.

15. A method of transferring a region of tissue, comprising:
securing a substrate upon the region of tissue to be removed from a first tissue site;
removing the region of tissue with the substrate attached from the first tissue site;
piercing a first support structure through a periphery of the region of tissue and substrate;
positioning a second support structure upon a second tissue site; and
positioning the first support structure upon the second tissue site such that the first support structure is positioned within the second support structure and the transferred region of tissue is located upon the second tissue site via the first support structure.

16. The method of claim 15 wherein securing the substrate upon the region of tissue comprises securing the substrate upon a nipple-areolar complex.

17. The method of claim 15 wherein securing the substrate upon the region of tissue comprises adhering the substrate to the region of tissue.

18. The method of claim 15 wherein removing the region of tissue comprises cutting the region of tissue from the first tissue site.

19. The method of claim 15 wherein piercing the first support structure comprises piercing a first plurality of piercing features projecting from a first side of the first support structure into the periphery of the region of tissue and substrate.

20. The method of claim 15 wherein positioning the second support structure further comprises removing the epidermis from the second tissue site.

21. The method of claim 15 wherein positioning the first support structure upon the second tissue site comprises piercing a first plurality of piercing features extending from the first support structure into the second tissue site.

22. The method of claim 15 further comprising securing the first support structure and the second support structure upon the second tissue site.

23. A tissue transfer apparatus, comprising:
a support structure having an elliptical or circular configuration sized to encircle a region of tissue;
a plurality of piercing features projecting from a first side of the support structure;
a transfer substrate configured to approximate a size of the region of tissue when positioned upon the region of tissue.

24. The apparatus of claim 23 wherein the support structure is configured to encircle the region of tissue comprising a nipple-areolar complex.

25. The apparatus of claim 23 wherein the support structure comprises a ring-shaped member.

26. The apparatus of claim 23 wherein the plurality of piercing features comprises needles.

27. The apparatus of claim 23 wherein the plurality of piercing features comprise flat needles positioned orthogonally from an inner or outer diameter of the support structure.

28. The apparatus of claim 23 further comprising a hub having a central member with one or more central piercing features projecting therefrom and one or more guide members extending from the central member, wherein the hub is configured to slidingly mate within the support structure via the one or more guide members.

29. The apparatus of claim 23 wherein the one or more central piercing features comprise needles.

30. The apparatus of claim 23 further comprising a recipient site template configured to receive the support structure when the recipient site template is positioned upon a recipient site.

31. The apparatus of claim 23 wherein the transfer substrate comprises an adhesive substrate.

32. A method of transferring a nipple-areola complex, comprising:
removing a nipple from a first tissue site;
securing the nipple to a central member via one or more central piercing features projecting from the central member;
removing at least a portion of areola from the first tissue site;
positioning the nipple within the portion of areola via the central member;
piercing a support structure through a periphery of the areola such that the support structure and central member are aligned with one another;
positioning a recipient site template upon a second tissue site; and
securing the support structure and central member to the second tissue site.

33. The method of claim 32 further comprising securing a substrate upon the portion of areola prior to removing at least the portion of areola.

34. The method of claim 32 wherein the central member is aligned within the support structure via one or more guide members extending from the central member.

35. The method of claim 32 wherein positioning the recipient site template further comprises removing an epidermis from the second tissue site.

36. The method of claim 32 wherein securing the nipple-areola complex comprises piercing the plurality of piercing features through the recipient site template and into the second tissue site.

37. The method of claim 32 further comprising positioning a second substrate upon the nipple-areola complex and recipient site template.
